(19) United States
(12) Patent Application Publication
(10) Pub. No.: US 2026/0301167 A1
(43) Pub. Date: Oct. 1, 2026

(54) OCULAR ZONE SEGMENTATION WITH PATHOLOGIC FEATURE INTEGRATION

(71) Applicant: The Cleveland Clinic Foundation, Cleveland, OH (US)

(72) Inventors: Justis P Ehlers, Shaker Heights, OH (US); Sunil K Srivastava, Shaker Heights, OH (US)

(21) Appl. No.: 19/479,257

(22) PCT Filed: Apr. 29, 2024
(86) PCT No.: PCT/US2024/026849

§ 371 Date: Oct. 27, 2025

Related U.S. Application Data
(60) Provisional application No. 63/462,950, filed on Apr. 28, 2023.

(51) Int. Cl. G06T 7/00; G06N 20/00; G06T 7/11; G06T 7/62

(52) U.S. Cl. CPC G06T 7/0012; G06N 20/00; G06T 7/11; G06T 7/62; G06T 2207/20081; G06T 2207/30041

(57) ABSTRACT

One or more computing devices, systems and/or methods are provided. In an example, one or more images of an eye of a person may be received. A first machine learning model may be used to generate a first representation indicative of a first ocular zone based upon the one or more images. A second machine learning model may be used to generate a second representation indicative of one or more second ocular zones of the eye based upon the one or more images. An ocular segmentation profile indicative of first segment corresponding to the first ocular zone of the eye may be generated based upon the first representation and/or the second representation.

### RELATED APPLICATION

This application is a national stage application, filed under 35 U.S.C. § 371, of International Patent Application No. PCT/US24/026849, filed on Apr. 29, 2024, which claims priority to U.S. Provisional Patent Application No. 63/462,950, filed on Apr. 28, 2023, entitled “Multiple Model Integration and Implementation Systems for Multi-Layer Retinal Segmentation: Single Line, Multi-Line, Region-of-Interest, and Pathologic Feature integration.” U.S. Provisional Patent Application No. 63/462,950 and International Patent Application No. PCT/US24/026849 are incorporated herein by reference in their entirety.

### TECHNICAL FIELD

The present disclosure relates to evaluating ocular images.

### BACKGROUND

Eye diseases include various conditions affecting vision, including age-related macular degeneration (AMD), diabetic retinopathy, and other conditions. Early detection of eye diseases through testing is important for managing these conditions effectively. Detecting AMD in its early stages allows for interventions that may slow its progression and preserve vision.

Similarly, early detection of diabetic retinopathy enables timely treatment to prevent and/or minimize vision loss.

### SUMMARY

In accordance with the present disclosure, one or more computing devices, systems and/or methods are provided. In an example, a method is provided. One or more images of an eye of a person may be received. A first machine learning model may be used to generate a first representation indicative of a first ocular zone based upon the one or more images. A second machine learning model may be used to generate a second representation indicative of one or more second ocular zones of the eye based upon the one or more images. An ocular segmentation profile indicative of a plurality of segments corresponding to a plurality of ocular zones of the eye may be generated based upon the first representation and/or the second representation.

In an example, an ocular zone segmentation module is provided. The ocular zone segmentation module is configured to (i) receive one or more images of an eye of a person, (ii) use a first machine learning model to generate a first representation indicative of a first ocular zone of the eye based upon the one or more images, (iii) use a second machine learning model to generate a second representation indicative of one or more second ocular zones of the eye based upon the one or more images, and/or (iv) generate, based upon the first representation and the second representation, an ocular segmentation profile indicative of a plurality of segments corresponding to a plurality of ocular zones of the eye.

In an example, a system is provided. The system comprises the ocular zone segmentation module and a feature extraction module configured to identify one or more pathological features of the eye based upon at least one of the first representation, the second representation or the one or more images. The ocular zone segmentation module may generate the ocular segmentation profile based upon the one or more pathological features.

In an example, a method is provided. One or more images of an eye of a person may be received. A first machine learning model may be used to generate a first representation indicative of a first ocular zone based upon the one or more images. A second machine learning model may be used to generate a second representation indicative of one or more second ocular zones of the eye based upon the one or more images. An ocular segmentation profile indicative of a first segment corresponding to the first ocular zone of the eye may be generated based upon the first representation and/or the second representation.

### DESCRIPTION OF THE DRAWINGS

While the techniques presented herein may be embodied in alternative forms, the particular embodiments illustrated in the drawings are only a few examples that are supplemental of the description provided herein. These embodiments are not to be interpreted in a limiting manner, such as limiting the claims appended hereto.

FIG. 1 is an illustration of a scenario involving various examples of networks that may connect servers and clients.

FIG. 2 is an illustration of a scenario involving an example configuration of a server that may utilize and/or implement at least a portion of the techniques presented herein.

FIG. 3 is an illustration of a scenario involving an example configuration of a client that may utilize and/or implement at least a portion of the techniques presented herein.

FIG. 4A is a component block diagram illustrating training a machine learning model, in accordance with some embodiments.

FIG. 4B is a component block diagram illustrating training a machine learning model, in accordance with some embodiments.

FIG. 4C is a component block diagram illustrating training a machine learning model, in accordance with some embodiments.

FIG. 4D is a component block diagram illustrating training a machine learning model, in accordance with some embodiments.

FIG. 5 is a component block diagram illustrating a system comprising a segmentation module interacting with a feature extraction module, in accordance with some embodiments.

FIG. 6A is a component block diagram illustrating a segmentation task being performed to derive a segmentation representation, in accordance with some embodiments.

FIG. 6B is a component block diagram illustrating generation of a segment of an ocular segmentation profile based upon a segmentation representation and one or more supplemental segmentation representations, in accordance with some embodiments.

FIG. 6C illustrates an example interaction between a trained single-line model and a trained region of interest model, in accordance with some embodiments.

FIG. 7 illustrates an example representation of an ocular segmentation profile, in accordance with some embodiments.

FIG. 8A is a component block diagram illustrating identification of one or more pathological features, in accordance with some embodiments.

FIG. 8B is a component block diagram illustrating identification of one or more pathological features, in accordance with some embodiments.

FIG. 8C is a component block diagram illustrating identification of one or more pathological features, in accordance with some embodiments.

FIG. 9A illustrates an example interaction between an ocular segmentation profile and a set of pathological features, in accordance with some embodiments.

FIG. 9B illustrates an example interaction between an ocular segmentation profile and a set of pathological features, in accordance with some embodiments.

FIG. 10 illustrates an example representation of at least a portion of an ocular report, in accordance with some embodiments.

FIG. 11 is a flow chart illustrating an example method, in accordance with some embodiments.

FIG. 12 is an illustration of a scenario featuring an example non-transitory machine readable medium in accordance with one or more of the provisions set forth herein.

### DETAILED DESCRIPTION

Subject matter will now be described more fully hereinafter with reference to the accompanying drawings, which form a part hereof, and which show, by way of illustration, specific example embodiments. This description is not intended as an extensive or detailed discussion of known concepts. Details that are known generally to those of ordinary skill in the relevant art may have been omitted, or may be handled in summary fashion.

The following subject matter may be embodied in a variety of different forms, such as methods, devices, components, and/or systems. Accordingly, this subject matter is not intended to be construed as limited to any example embodiments set forth herein. Rather, example embodiments are provided merely to be illustrative. Such embodiments may, for example, take the form of hardware, software, firmware, medicine, clothing design, or any combination thereof.

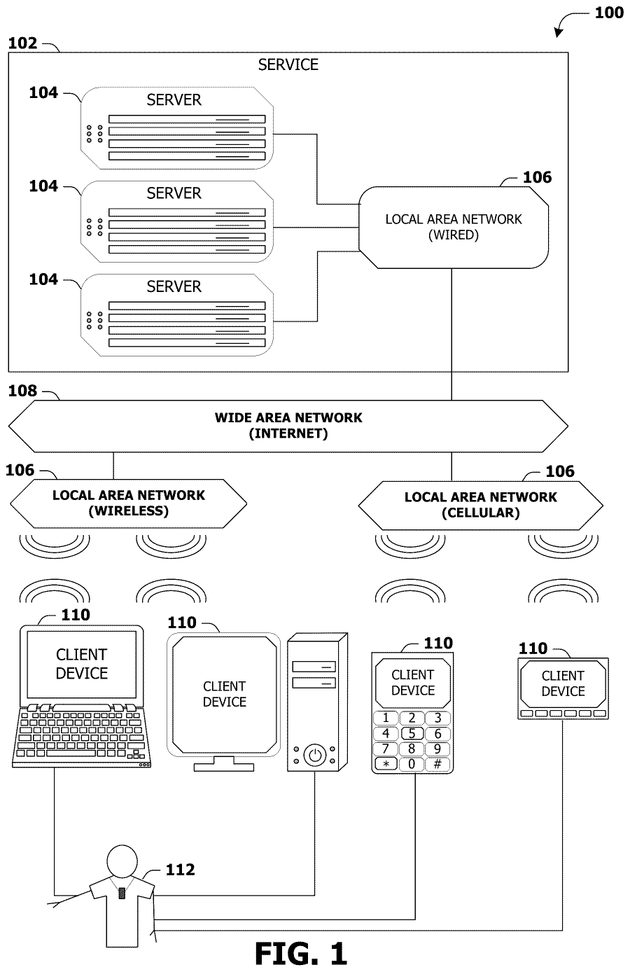

FIG. 1 is an interaction diagram of a scenario 100 illustrating a service 102 provided by a set of servers 104 to a set of client devices 110 via various types of networks. The servers 104 and/or client devices 110 may be capable of transmitting, receiving, processing, and/or storing many types of signals, such as in memory as physical memory states.

In the scenario 100 of FIG. 1, the service 102 may be accessed via a wide area network 108 (WAN) by a user 112 of one or more client devices 110, such as a portable media player (e.g., an electronic text reader, an audio device, or a portable gaming, exercise, or navigation device); a portable communication device (e.g., a camera, a phone, a wearable or a text chatting device); a workstation; and/or a laptop form factor computer. The respective client devices 110 may communicate with the service 102 via various connections to the wide area network 108.

One or more client devices 110 may comprise a cellular communicator and may communicate with the service 102 by connecting to the wide area network 108 via a wireless local area network 106 (LAN) provided by a cellular provider.

Alternatively and/or additionally, one or more client devices 110 may communicate with the service 102 by connecting to the wide area network 108 via a wireless local area network 106 provided by a location such as the user's home or workplace. The wireless local area network 106 may, for example, be a WiFi (Institute of Electrical and Electronics Engineers (IEEE) Standard 802.11) network or a Bluetooth (IEEE Standard 802.15.1) personal area network.

It may be appreciated that the servers 104 and the client devices 110 may communicate over various types of networks. Exemplary types of networks that may be accessed by the servers 104 and/or client devices 110 include mass storage, such as network attached storage (NAS), a storage area network (SAN), or other forms of computer or machine readable media.

The servers 104 of the service 102 may be interconnected directly, or through one or more other networking devices, such as routers, switches, and/or repeaters. The servers 104 may utilize a variety of physical networking protocols, such as Ethernet and/or Fiber Channel, and/or logical networking protocols, such as variants of an Internet Protocol (IP), a Transmission Control Protocol (TCP), and/or a User Datagram Protocol (UDP).

The servers 104 of the service 102 may be internally connected via a local area network 106. The local area network 106 may be organized according to one or more network architectures, such as server/client, peer-to-peer, and/or mesh architectures, and/or a variety of roles, such as administrative servers, authentication servers, security monitor servers, data stores for objects such as files and databases, business logic servers, time synchronization servers, and/or front-end servers providing a user-facing interface for the service 102.

The local area network 106 may be a wired network where network adapters on the respective servers 104 are interconnected via cables (e.g., coaxial and/or fiber optic cabling), and may be connected in various topologies (e.g., buses, token rings, meshes, and/or trees). The local area network 106 may include, e.g., analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as may be known to those skilled in the art.

Alternatively and/or additionally, the local area network 106 may comprise one or more sub-networks, such as may employ differing architectures, may be compliant or compatible with differing protocols and/or may interoperate within the local area network 106. Additionally, a variety of local area networks 106 may be interconnected; e.g., a router may provide a link between otherwise separate and independent local area networks 106.

In the scenario 100 of FIG. 1, the local area network 106 of the service 102 is connected to a wide area network 108 that allows the service 102 to exchange data with other services 102 and/or client devices 110. The wide area network 108 may encompass various combinations of devices with varying levels of distribution and exposure, such as a public wide-area network (e.g., the Internet) and/or a private network (e.g., a virtual private network (VPN) of a distributed enterprise).

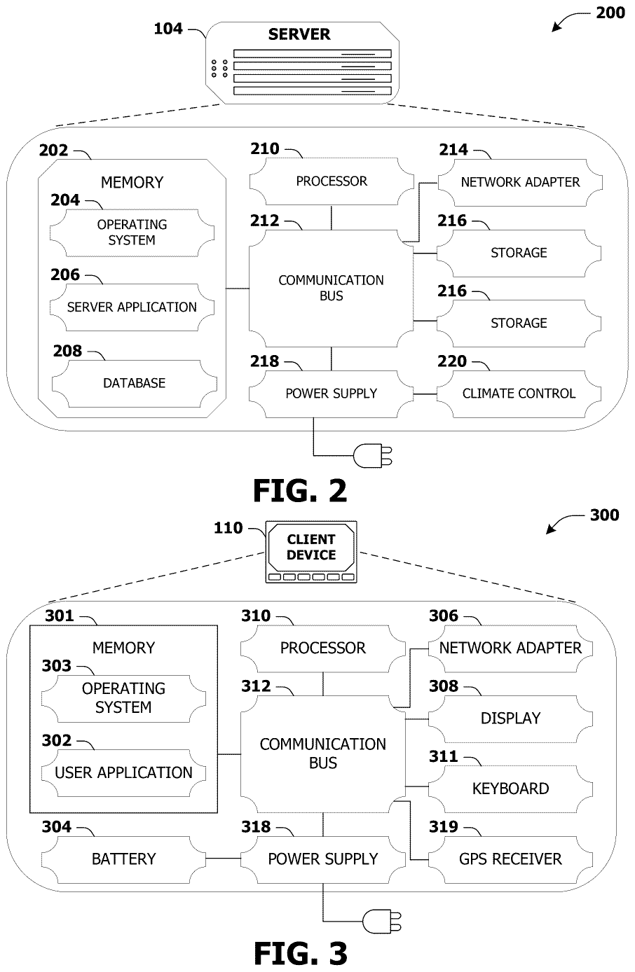

FIG. 2 presents a schematic architecture diagram 200 of a server 104 that may utilize at least a portion of the techniques provided herein. Such a server 104 may vary widely in configuration or capabilities, alone or in conjunction with other servers, in order to provide a service such as the service 102.

The server 104 may comprise a variety of peripheral components, such as a wired and/or wireless network adapter 214 connectible to a local area network and/or wide area network; one or more storage components 216, such as a hard disk drive, a solid-state storage device (SSD), a flash memory device, and/or a magnetic and/or optical disk reader.

The server 104 may comprise memory 202 storing various forms of applications, such as an operating system 204; one or more server applications 206, such as a hypertext transport protocol (HTTP) server, a file transfer protocol (FTP) server, or a simple mail transport protocol (SMTP) server; and/or various forms of data, such as a database 208 or a file system.

The server 104 may comprise one or more processors 210 that process instructions. The one or more processors 210 may optionally include a plurality of cores; one or more coprocessors, such as a mathematics coprocessor or an integrated graphical processing unit (GPU); and/or one or more layers of local cache memory.

The server 104 may comprise a mainboard featuring one or more communication buses 212 that interconnect the processor 210, the memory 202, and various peripherals, using a variety of bus technologies, such as a variant of a serial or parallel AT Attachment (ATA) bus protocol; a Uniform Serial Bus (USB) protocol; and/or Small Computer System Interface (SCI) bus protocol. In a multibus scenario, a communication bus 212 may interconnect the server 104 with at least one other server.

The server 104 may operate in various physical enclosures, such as a desktop or tower, and/or may be integrated with a display as an “all-in-one” device. The server 104 may be mounted horizontally and/or in a cabinet or rack, and/or may simply comprise an interconnected set of components.

The server 104 may provide power to and/or receive power from another server and/or other devices. The server 104 may comprise a dedicated and/or shared power supply 218 that supplies and/or regulates power for the other components. The server 104 may comprise a shared and/or dedicated climate control unit 220 that regulates climate properties, such as temperature, humidity, and/or airflow.

The server 104 may include one or more other components that are not shown in the schematic diagram 200 of FIG. 2, such as a display; a display adapter, such as a graphical processing unit (GPU); input peripherals, such as a keyboard and/or mouse; and a flash memory device that may store a basic input/output system (BIOS) routine that facilitates booting the server 104 to a state of readiness. A plurality of such servers 104 may be configured and/or adapted to utilize at least a portion of the techniques presented herein.

FIG. 3 presents a schematic architecture diagram 300 of a client device 110 whereupon at least a portion of the techniques presented herein may be implemented. Such a client device 110 may vary widely in configuration or capabilities, in order to provide a variety of functionality to a user such as the user 112.

The client device 110 may comprise memory 301 storing various forms of applications, such as an operating system 303; one or more user applications 302, such as document applications, media applications, file and/or data access applications, communication applications such as web browsers and/or email clients, utilities, and/or games; and/or drivers for various peripherals.

In some examples, as a user 112 interacts with a software application on a client device 110 (e.g., an instant messenger and/or electronic mail application), descriptive content in the form of signals or stored physical states within memory (e.g., an email address, instant messenger identifier, phone number, postal address, message content, date, and/or time) may be identified.

In such examples, descriptive content may be stored, typically along with contextual content. For example, the source of an email address (e.g., a communication received from another user via an instant messenger application) may be stored as contextual content associated with the email address. Contextual content, therefore, may identify circumstances surrounding receipt of an email address (e.g., the date or time that the email address was received), and may be associated with descriptive content. Contextual content, may, for example, be used to subsequently search for associated descriptive content. For example, a search for email addresses received from specific individuals, received via an instant messenger application or at a given date or time, may be initiated.

The client device 110 may comprise one or more processors 310 that process instructions. The one or more processors 310 may optionally include a plurality of cores; one or more coprocessors, such as a mathematics coprocessor or an integrated graphical processing unit (GPU); and/or one or more layers of local cache memory.

The client device 110 may comprise a dedicated and/or shared power supply 318 that supplies and/or regulates power for other components, and/or a battery 304 that stores power for use while the client device 110 is not connected to a power source via the power supply 318. The client device 110 may provide power to and/or receive power from other client devices.

The client device 110 may comprise a variety of peripheral components, such as a wired and/or wireless network adapter 306 connectible to a local area network and/or wide area network; one or more output components, such as a display 308 coupled with a display adapter (optionally including a graphical processing unit (GPU)), a sound adapter coupled with a speaker, and/or a printer; input devices for receiving input from the user, such as a keyboard 311, a mouse, a microphone, a camera, and/or a touch-sensitive component of the display 308; and/or environmental sensors, such as a global positioning system (GPS) receiver 319 that detects the location, velocity, and/or acceleration of the client device 110, a compass, accelerometer, and/or gyroscope that detects a physical orientation of the client device 110.

The client device 110 may comprise a mainboard featuring one or more communication buses 312 that interconnect the processor 310, the memory 301, and various peripherals, using a variety of bus technologies, such as a variant of a serial or parallel AT Attachment (ATA) bus protocol; the Uniform Serial Bus (USB) protocol; and/or the Small Computer System Interface (SCI) bus protocol.

The client device 110 may include one or more other components that are not shown in the schematic architecture diagram 300 of FIG. 3, such as one or more storage components, such as a hard disk drive, a solid-state storage device (SSD), a flash memory device, and/or a magnetic and/or optical disk reader; and/or a flash memory device that may store a basic input/output system (BIOS) routine that facilitates booting the client device 110 to a state of readiness. In some examples, the client device 110 may include a climate control unit that regulates climate properties, such as temperature, humidity, and airflow.

The client device 110 may include one or more servers that may locally serve the client device 110 and/or other client devices of the user 112 and/or other individuals. For example, a locally installed webserver may provide web content in response to locally submitted web requests. Many such client devices 110 may be configured and/or adapted to utilize at least a portion of the techniques presented herein.

The client device 110 may serve the user in a variety of roles, such as a workstation, kiosk, media player, gaming device, and/or appliance. The client device 110 may therefore be provided in a variety of form factors, such as a desktop or tower workstation; an “all-in-one” device integrated with a display 308; a laptop, tablet, convertible tablet, or palmtop device; a wearable device mountable in a headset, eyeglass, earpiece, and/or wristwatch, and/or integrated with an article of clothing; and/or a component of a piece of furniture, such as a tabletop, and/or of another device, such as a vehicle or residence.

One or more devices and/or techniques for evaluating ocular images of persons are provided. In some examples, a segmentation module may receive one or more images of an eye of a person. The segmentation module may use multiple machine learning models to perform multiple segmentation tasks associated with a first ocular zone of the eye on the one or more images to produce a plurality of segmentation representations. A feature extraction module may use one or more trained feature extraction models to identify one or more pathological features of the eye. The plurality of segmentation representations may be used in conjunction with the one or more pathological features to generate an ocular segmentation profile indicative of a first segment corresponding to the first ocular zone (identifying where the first ocular zone resides relative to one or more other ocular zones, for example) and/or other segments corresponding to other ocular zones of the eye. Using one or more of the techniques provided herein may provide for improved accuracy of the first segment corresponding to the first ocular zone, such as due, at least in part, to the first segment accounting for the one or more pathological features and/or the multiple machine learning models capturing different contexts (e.g., global context, local context, etc.) associated with the first ocular zone.

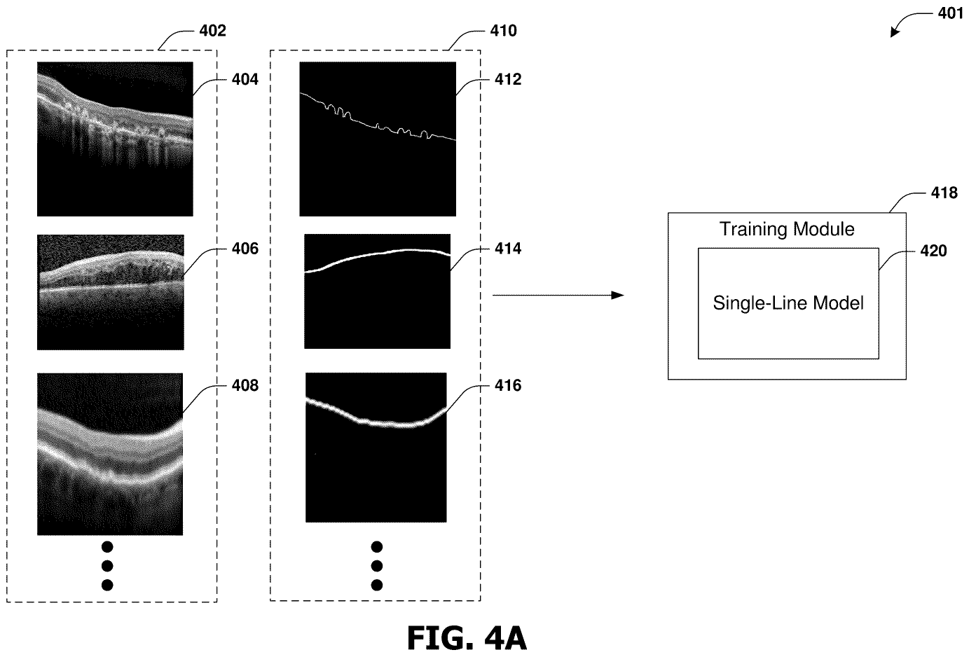

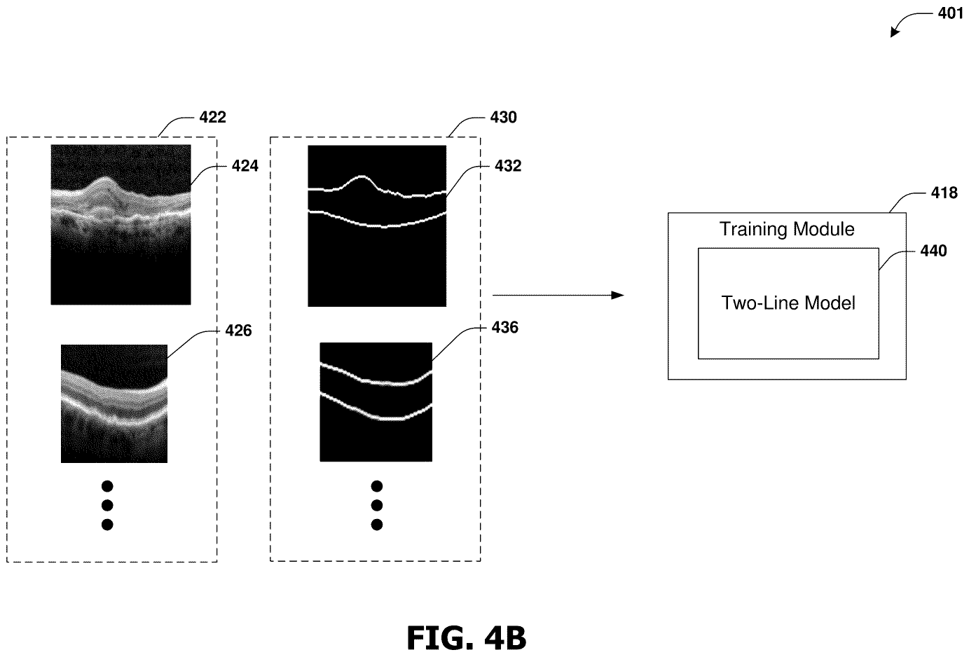

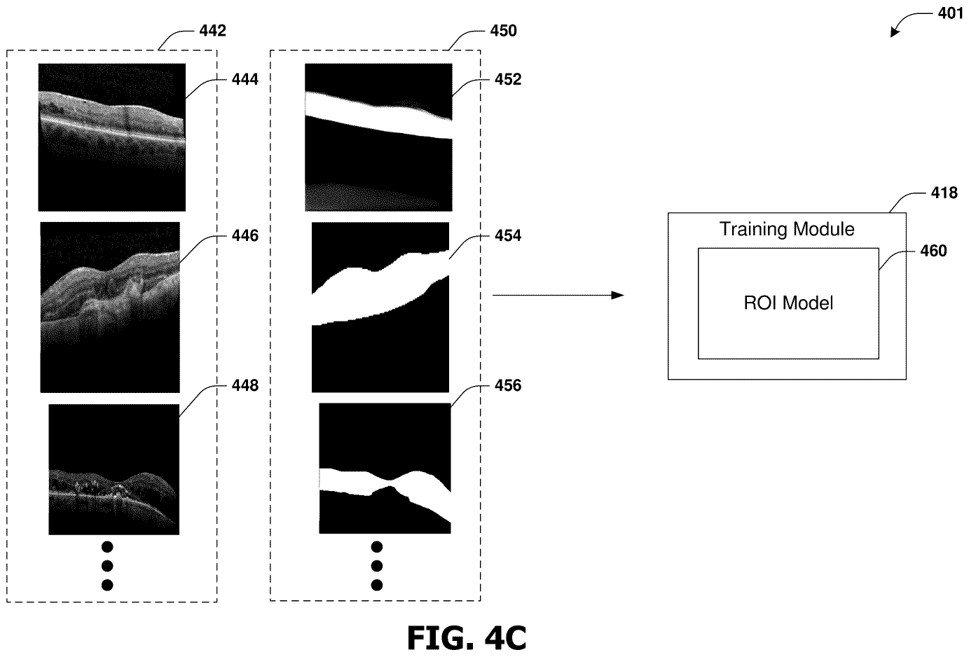

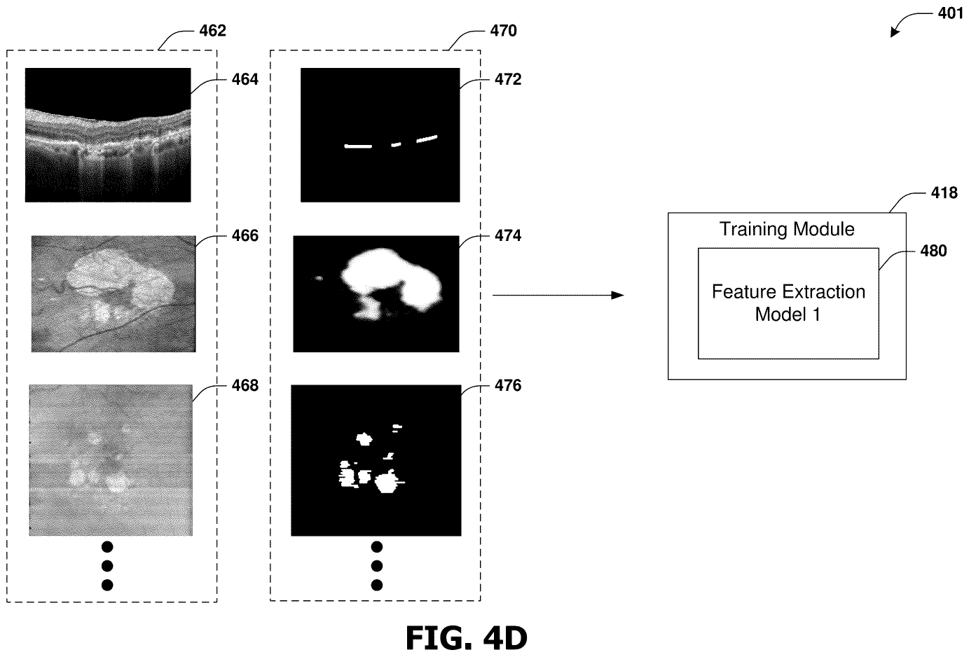

FIGS. 4A-4D illustrate a machine learning model training system 401 training machine learning models of a multi-model segmentation system with integrated feature extraction, in accordance with some embodiments. FIG. 4A illustrates using a training module 418 to train a machine learning model to generate a trained single-line model 420. In some examples, the training module 418 trains the machine learning model to generate the trained single-line model 420 using a first plurality of images 402 and/or first label information 410 associated with the first plurality of images 402. The first plurality of images 402 may comprise ocular images of a plurality of persons (e.g., persons that are diagnosed with one or more eye conditions and/or persons that are not diagnosed with any eye condition). In some examples, the first plurality of images 402 may comprise optical coherence tomography (OCT) images (e.g., OCT Brightness scans (B-scans) and/or Amplitude scans (A-scans)), such as spectral domain OCT (SD-OCT) images (e.g., SD-OCT B-scans and/or A-scans). In some examples, an OCT B-scan may comprise a cross-sectional image of a tissue of interest. An OCT B-scan may be a combination of a plurality of OCT A-scans (e.g., the sum of OCT A-scans) which creates the cross-sectional image of a tissue of interest. In some examples, the first plurality of images 402 may be adjusted (prior to being used for machine learning model training, for example), such as at least one of resized, cropped, compressed, etc., such that images of the first plurality of images 402 have at least one of the same size, the same number of pixels, the same dimensions, etc. In an example, each image of one, some or all of the first plurality of images 402 may be automatically adjusted to have the same size (e.g., 128×128 pixels, 512×512 pixels, and/or 256×256 pixels).

In some examples, the trained single-line model 420 is trained to perform a first segmentation task associated with a first ocular zone (e.g., merely the first ocular zone). For example, the first segmentation task may comprise identifying the first ocular zone within an image (e.g., distinguish a segment of the image that corresponds to the first ocular zone from the rest of the image). In an example, the first ocular zone may correspond to an ocular layer of a person's eye. The first ocular zone may correspond to a boundary of (and/or at least a portion of) at least one of an internal limiting membrane (ILM), a Bruch's membrane (BM), a retinal pigment epithelium (RPE), an outer nuclear layer (ONL), an ellipsoid zone (EZ), photoreceptor outer segments (POS), an external limiting membrane (ELM), an outer plexiform layer (OPL), an inner nuclear layer (INL), an inner plexiform layer (IPL), a ganglion cell layer (GCL), a retinal nerve fiber layer (RNFL), etc.

In some examples, the first label information 410 (e.g., ground truth information) may identify respective segments of the first plurality of images 402 that correspond to the first ocular zone. The first plurality of images 402 may comprise an image 404, an image 406, an image 408 and/or one or more other images. The first label information 410 may comprise (i) a label 412 (e.g., a ground truth segment) identifying a segment, of the image 404, that corresponds to the first ocular zone, (ii) a label 414 identifying a segment, of the image 406, that corresponds to the first ocular zone and/or (iii) a label 416 identifying a segment, of the image 408, that corresponds to the first ocular zone.

FIG. 4B illustrates training a machine learning model to generate a trained two-line model 440. In some examples, the training module 418 trains the machine learning model to generate the trained two-line model 440 using a second plurality of images 422 and/or second label information 430 associated with the second plurality of images 422. The second plurality of images 422 may comprise ocular images of a plurality of persons (e.g., persons that are diagnosed with one or more eye conditions and/or persons that are not diagnosed with any eye condition). For example, the second plurality of images 422 may comprise OCT images (e.g., OCT B-scans and/or A-scans), such as SD-OCT images (e.g., SD-OCT B-scans and/or A-scans). In some examples, the second plurality of images 422 may be adjusted (prior to being used for machine learning model training, for example), such as at least one of resized, cropped, compressed, etc., such that images of the second plurality of images 422 have at least one of the same size, the same number of pixels, the same dimensions, etc.

In some examples, the trained two-line model 440 is trained to perform a second segmentation task associated with one or more second ocular zones. For example, the second segmentation task may comprise identifying the one or more second ocular zones within an image (e.g., distinguish one or more segments of the image that correspond to the one or more second ocular zones from the rest of the image). In an example, the one or more second ocular zones may correspond to one or more ocular layers of a person's eye. In some examples, the one or more second ocular zones comprise the first ocular zone and a second ocular zone. The second ocular zone may correspond to a boundary of (and/or at least a portion of) at least one of an ILM, a BM, a RPE, an ONL, an EZ, POS, ELM, an OPL, an INL, an IPL, a GCL, a RNFL, etc.

In some examples, the second label information 430 may identify respective segments of the second plurality of images 422 that correspond to the one or more second ocular zones (e.g., the first ocular zone and the second ocular zone). The second plurality of images 422 may comprise an image 424, an image 426 and/or one or more other images. The second label information 430 may comprise (i) a label 432 identifying one or more segments, of the image 424, that corresponds to the one or more second ocular zones (e.g., the label 432 may identify a segment corresponding to the first ocular zone and a segment corresponding to the second ocular zone) and/or (ii) a label 436 identifying one or more segments, of the image 426, that corresponds to the one or more second ocular zones (e.g., the label 436 may identify a segment corresponding to the first ocular zone and a segment corresponding to the second ocular zone).

FIG. 4C illustrates training a machine learning model to generate a trained region of interest (ROI) model 460. In some examples, the training module 418 trains the machine learning model to generate the trained region of interest model 460 using a third plurality of images 442 and/or third label information 450 associated with the third plurality of images 442. The third plurality of images 442 may comprise ocular images of a plurality of persons (e.g., persons that are diagnosed with one or more eye conditions and/or persons that are not diagnosed with any eye condition). For example, the third plurality of images 442 may comprise OCT images (e.g., OCT B-scans and/or A-scans), such as SD-OCT images (e.g., SD-OCT B-scans and/or A-scans). In some examples, the third plurality of images 442 may be adjusted (prior to being used for machine learning model training, for example), such as at least one of resized, cropped, compressed, etc., such that images of the third plurality of images 442 have at least one of the same size, the same number of pixels, the same dimensions, etc.

In some examples, the trained region of interest model 460 is trained to perform a third segmentation task associated with the one or more second ocular zones. For example, the third segmentation task may comprise identifying a first region of interest (associated with the one or more second ocular zones) within an image (e.g., distinguish a segment of the image that corresponds to the first region of interest from the rest of the image). The first region of interest may correspond to a region between the first ocular zone and the second ocular zone.

In some examples, the third label information 450 may identify respective segments of the third plurality of images 442 that correspond to the first region of interest (between the first ocular zone and the second ocular zone). The third plurality of images 442 may comprise an image 444, an image 446, an image 448 and/or one or more other images. The third label information 450 may comprise (i) a label 452 identifying a segment, of the image 444, that corresponds the first region of interest between the first ocular zone and the second ocular zone, (ii) a label 454 identifying a segment, of the image 446, that corresponds the first region of interest between the first ocular zone and the second ocular zone, and/or (ii) a label 456 identifying a segment, of the image 448, that corresponds the first region of interest between the first ocular zone and the second ocular zone.

FIG. 4D illustrates training a machine learning model to generate a first trained feature extraction model 480. In some examples, the training module 418 trains the machine learning model to generate the first trained feature extraction model 480 using a fourth plurality of images 462 and/or fourth label information 470 associated with the fourth plurality of images 462. The fourth plurality of images 462 may comprise ocular images of a plurality of persons (e.g., persons that are diagnosed with one or more eye conditions and/or persons that are not diagnosed with any eye condition). For example, the fourth plurality of images 462 may comprise OCT images (e.g., OCT B-scans and/or A-scans), such as SD-OCT images (e.g., SD-OCT B-scans and/or A-scans). Alternatively and/or additionally, the fourth plurality of images 462 may comprise en face images (e.g., en face OCT images). In some examples, a plurality of OCT images (e.g., a plurality of OCT B-scans) are reconstructed to generate an en face image of the fourth plurality of images 462. In some examples, the fourth plurality of images 462 may be adjusted (prior to being used for machine learning model training, for example), such as at least one of resized, cropped, compressed, etc., such that images of the fourth plurality of images 462 have at least one of the same size, the same number of pixels, the same dimensions, etc.

In some examples, the first trained feature extraction model 480 is trained to perform a feature extraction task comprising identifying one or more pathological features within an image (e.g., distinguish one or more segments of the image that correspond to the one or more pathological features from the rest of the image). In some examples, the first trained feature extraction model 480 is trained to identify one or more first pathological feature types. In some examples, the one or more first pathological feature types comprise at least one of intraretinal fluid (IRF) (e.g., longitudinal IRF), subretinal fluid (SRF) (e.g., longitudinal SRF), Subretinal material (SRMat), subretinal hyperreflective material (SHRM), geographic atrophy (GA), drusen (e.g., extracellular deposits of lipids, proteins, and/or cellular debris found within one or more layers of a retina), inflammatory debris, inflammatory lesion, hypertransmission defect, hypotransmission defect, cystic fluid, general fluid, sub-RPE fluid, a lesion, etc.

In some examples, the fourth label information 470 may identify respective segments of the fourth plurality of images 462 that correspond to pathological features of the one or more first types of pathological features. The fourth plurality of images 462 may comprise an image 464 (e.g., an OCT B-scan), an image 466 (e.g., an en face OCT image), an image 468 (e.g., an en face OCT image) and/or one or more other images. In an example, the one or more first types of pathological features comprise geographic atrophy (GA). The fourth label information 470 may comprise (i) a label 472 identifying one or more segments, of the image 464, corresponding to GA, (ii) a label 474 identifying one or more segments, of the image 466, corresponding to GA, and/or (iii) a label 476 identifying one or more segments, of the image 468, corresponding to GA.

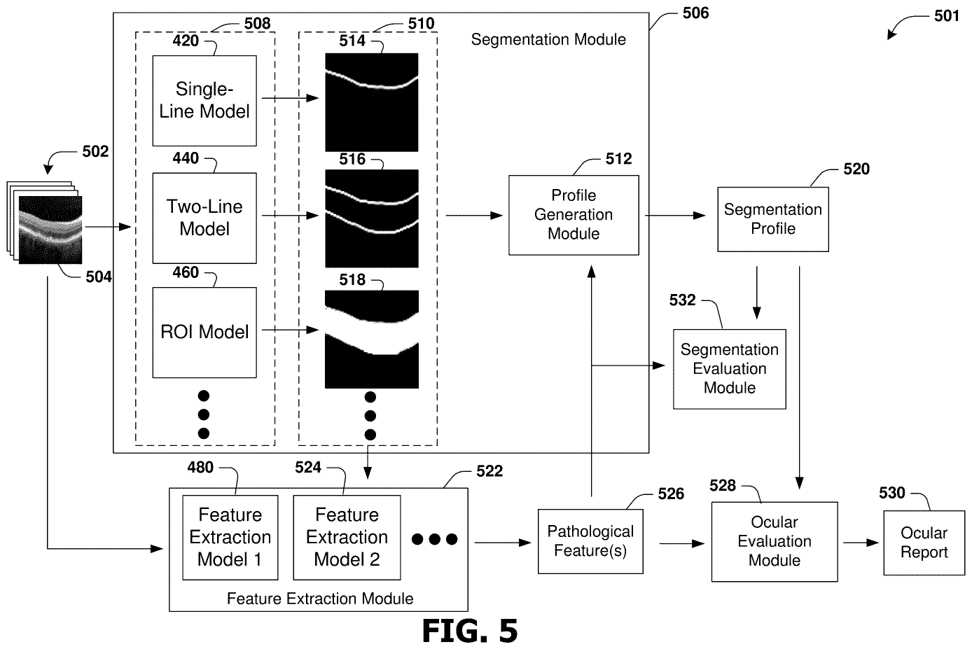

FIG. 5 illustrates a system 501 comprising a segmentation module 506 interacting with a feature extraction module 522 to evaluate a set of (one or more) images 502 of an eye of a person (e.g., a patient). In some examples, the set of images 502 may comprise one or more OCT images (e.g., OCT B-scans and/or A-scans), such as SD-OCT images (e.g., SD-OCT B-scans and/or A-scans). Alternatively and/or additionally, the set of images 502 may comprise one or more en face images (e.g., en face OCT images).

The segmentation module 506 may comprise a multi-model machine learning segmentation system comprising a plurality of machine learning models 508 associated with a plurality of segmentation tasks. For example, the plurality of machine learning models 508 may comprise the trained single-line model 420 trained to perform the first segmentation task, the trained two-line model 440 trained to perform the second segmentation task, the trained region of interest model 460 trained to perform the third segmentation task and/or one or more other machine learning models trained to perform one or more other segmentation tasks. The plurality of machine learning models 508 may be used to perform respective segmentation tasks of the plurality of segmentation tasks on one, some or all of the set of images 502 to generate a plurality of segmentation representations 510.

For example, the trained single-line model 420 may perform the first segmentation task on a first image 504 (e.g., an OCT B-scan of at least a portion of the eye of the person) of the set of images 502 to generate a first segmentation representation 514 (e.g., a line segmentation) indicative of the first ocular zone. For example, the first segmentation representation 514 may comprise a representation of the first ocular zone relative to the first image 504 (e.g., the first segmentation representation 514 may be indicative of a segment, of the first image 504, corresponding to the first ocular zone). In an example, the trained single-line model 420 may be configured such that the first segmentation representation 514 is generated to include merely a line segmentation (e.g., merely a single line segmentation) for the first ocular zone (e.g., merely the first ocular zone).

Alternatively and/or additionally, the trained two-line model 440 may perform the second segmentation task on the first image 504 to generate a second segmentation representation 516 (e.g., one or more line segmentations) indicative of the one or more second ocular zones. For example, the second segmentation representation 516 may comprise (i) a representation of the first ocular zone (of the one or more second ocular zones) relative to the first image 504 and/or (ii) a representation of the second ocular zone (of the one or more second ocular zones) relative to the first image 504 (e.g., the second segmentation representation 516 may be indicative of a segment, of the first image 504, corresponding to the first ocular zone and a segment, of the first image 504, corresponding to the second ocular zone). In an example, the trained two-line model 440 may be configured such that the second segmentation representation 516 is generated to include merely (i) a line segmentation for the first ocular zone (e.g., merely the first ocular zone) and (ii) a line segmentation for the second ocular zone (e.g., merely the second ocular zone).

Alternatively and/or additionally, the trained region of interest model 460 may perform the third segmentation task on the first image 504 to generate a third segmentation representation 518 (e.g., a region of interest segmentation) indicative of the one or more second ocular zones. For example, the third segmentation representation 518 may comprise a representation of the first region of interest (between the first ocular zone and the second ocular zone) relative to the first image 504 (e.g., the third segmentation representation 518 may be indicative of a segment, of the first image 504, corresponding to the first region of interest between the first ocular zone and the second ocular zone). In an example, the trained region of interest model 460 may be configured such that the third segmentation representation 518 is generated to include merely a region of interest segmentation for the first region of interest between the first ocular zone and the second ocular zone.

The plurality of segmentation representations 510 (e.g., the first segmentation representation 514, the second segmentation representation 516, the third segmentation representation 518 and/or one or more other representations) may be provided to a profile generation module 512 for use in generating an ocular segmentation profile 520 associated with the eye of the person. Alternatively and/or additionally, the plurality of segmentation representations 510 may be provided to the feature extraction module 522 for use in determining a set of (one or more) pathological features 526 of the eye. In some examples, each feature of one, some or all of the set of pathological features 526 may be associated with a defined impact on one or more ocular zones (e.g., one or more ocular layers of the eye of the person) and/or one or more retinal compartment zones (e.g., one or more retinal compartment layers of the eye of the person).

In some examples, the ocular segmentation profile 520 may be indicative of a plurality of segments corresponding to a plurality of ocular zones of the eye of the person. The plurality of segments may comprise line segmentations corresponding to boundaries of ocular zones and/or region of interest segmentations corresponding to regions associated with ocular zones. The plurality of ocular zones may comprise the first ocular zone, the second ocular zone and/or one or more other ocular zones. The plurality of segments may comprise a first segment (e.g., a line segmentation) corresponding to the first ocular zone, a second segment (e.g., a line segmentation) corresponding to the second ocular zone and/or other segments (e.g., line segmentations and/or region of interest segmentations) corresponding to other ocular zones of the eye. The plurality of ocular zones may comprise at least one of an ILM, a BM, a RPE, an ONL, an EZ, POS, ELM, an OPL, an INL, an IPL, a GCL, a RNFL, etc. of the eye of the person.

The profile generation module 512 may generate the first segment (corresponding to the first ocular zone) based upon one, some or all segmentation representations of the plurality of segmentation representations 510. In an example, the profile generation module 512 may combine some or all segmentation representations of the plurality of segmentation representations 510 to generate the first segment. Alternatively and/or additionally, the profile generation module 512 may use one segmentation representation of the plurality of segmentation representations 510 to generate a first portion of the first segment and/or use another segmentation representation of the plurality of segmentation representations 510 to generate a second portion of the first segment. Alternatively and/or additionally, the profile generation module 512 may use one segmentation representation of the plurality of segmentation representations 510 to generate one version of the first segment and/or may use another segmentation representation of the plurality of segmentation representations 510 to generate an updated version of the first segment.

Alternatively and/or additionally, the profile generation module 512 may determine one or more confidence scores associated with one or more segmentation representations of the plurality of segmentation representations 510, and may use the one or more confidence scores to determine (i) whether to use the one or more segmentation representations for generating the first segment corresponding to the first ocular zone and/or (ii) whether to generate and/or use one or more additional segmentation representations for use in generating the first segment corresponding to the first ocular zone. In an example, the segmentation module 506 may (i) generate one or more first segmentation representations using a subset of the plurality of machine learning models 508, (ii) evaluate the one or more first segmentation representations to determine one or more first confidence scores, and/or (iii) use the one or more first confidence scores to determine whether to generate one or more further segmentation representations (in addition to the one or more first segmentation representations) using one or more remaining machine learning models of the plurality of machine learning models 508. For example, the segmentation module 506 may compare the one or more first confidence scores with one or more thresholds and/or may (i) determine to generate the one or more further segmentation representations using the one or more remaining machine learning models based upon one, some or all of the one or more first confidence scores not meeting the one or more thresholds and/or (ii) determine not to generate the one or more further segmentation representations using the one or more remaining machine learning models and/or may determine to generate the first segment corresponding to the first ocular zone using (merely) the one or more first segmentation representations. In an example, the one or more first segmentation representations may comprise the first segmentation representation 514 and/or the second segmentation representation 516 and/or the one or more further segmentation representations may comprise the third segmentation representation 518. In an example, the one or more first segmentation representations may comprise the first segmentation representation 514 and/or the one or more further segmentation representations may comprise the second segmentation representation 516 and/or the third segmentation representation 518.

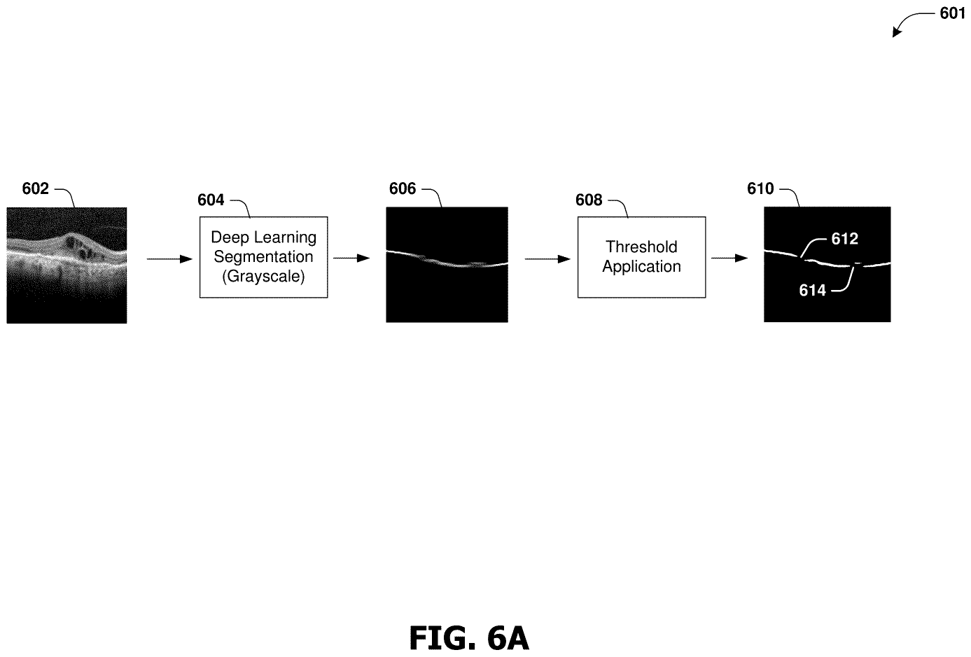

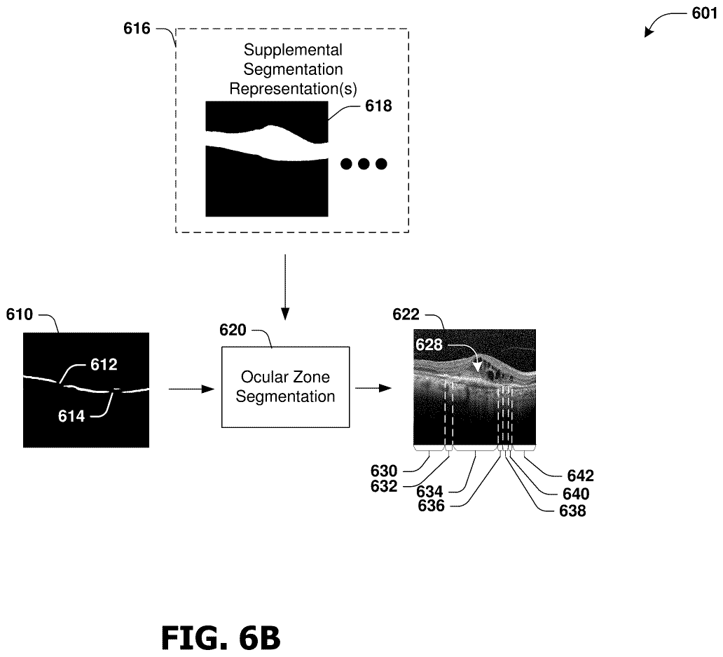

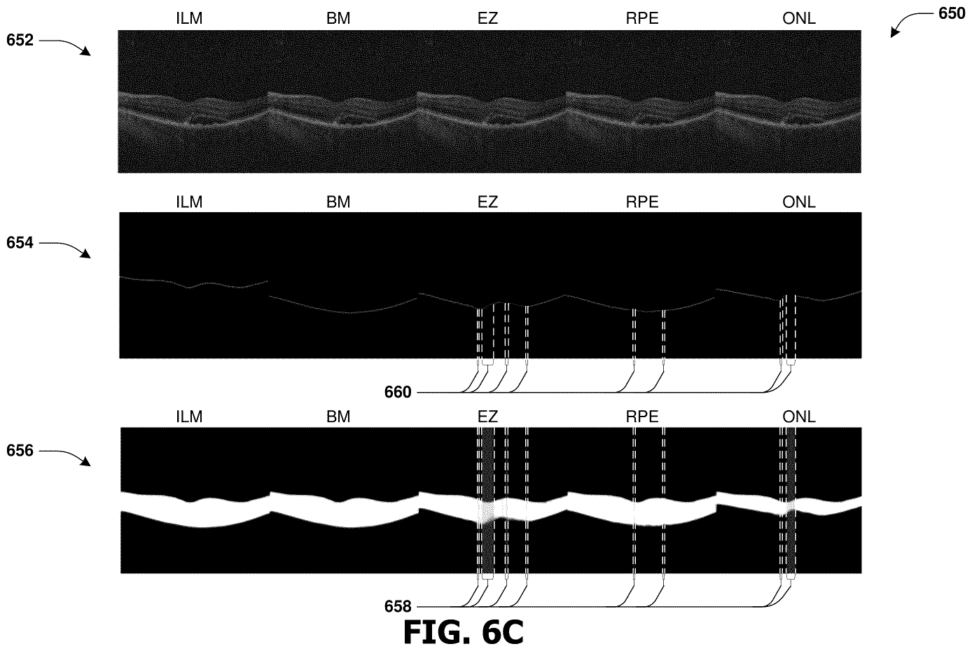

FIGS. 6A-6B illustrate a scenario 601 associated with performing a segmentation task to derive a segmentation representation of the plurality of segmentation representations 510 and/or using the segmentation representation in conjunction with one or more supplemental segmentation representations to generate a segment of the ocular segmentation profile 520.

In FIG. 6A, an image 602 (e.g., an OCT B-scan of at least a portion of the eye of the person) of the set of images 502 may be processed using a deep learning segmentation model 604 of the trained single-line model 420 to generate a grayscale segmentation representation 606 indicative of the first ocular zone. In some examples, the deep learning segmentation model 604 may (i) determine classification scores associated with whether pixels of the image 602 correspond to the first ocular zone, the classification scores including a first classification score corresponding to a first likelihood that a first pixel of the image 602 corresponds to the first ocular zone, a second classification score corresponding to a second likelihood that a first second of the image 602 corresponds to the first ocular zone, etc., (ii) determine grayscale pixel values based upon the classification scores (e.g., a grayscale pixel value may be a function of a classification score, where a higher value of the classification score may corresponds to a lower value of the grayscale pixel value that is closer to white and/or a lower value of the classification score may correspond to a higher value of the grayscale pixel value that is closer to black), and/or (iii) generate the grayscale segmentation representation 606 based upon the grayscale pixel values. In some examples, a threshold application module 608 of the trained single-line model 420 may apply a threshold to the grayscale segmentation representation 606 to generate a binary segmentation representation 610. For example, the threshold application module 608 may (i) compare a threshold classification score with classification scores represented by pixel values of the grayscale segmentation representation 606 to identify pixels associated with classification scores that meet (e.g., exceed) the threshold classification score and/or (ii) generate the binary segmentation representation 610 such that the identified pixels are set to a first color (e.g., white) and/or remaining pixels (e.g., pixels associated with classification scores that do not meet the threshold classification score) are set to a second color (e.g., black). Alternatively and/or additionally, the threshold application module 608 may (i) compare a threshold pixel value with pixel values of the grayscale segmentation representation 606 to identify pixels associated with classification scores that meet (e.g., exceed) the threshold pixel value and/or (ii) generate the binary segmentation representation 610 such that the identified pixels are set to the first color (e.g., white) and/or remaining pixels (e.g., pixels associated with pixel values that do not meet the threshold pixel value) are set to the second color (e.g., black). In some examples, the plurality of segmentation representations 510 may comprise one or more grayscale segmentation representations (e.g., the grayscale segmentation representation 606) and/or one or more binary segmentation representations (e.g., the binary segmentation representation 610).

In FIG. 6B, an ocular zone segmentation module 620 of the profile generation module 512 may process the binary segmentation representation 610 generated using the trained single-line model 420 in conjunction with a set of (one or more) supplemental segmentation representations 616 of the plurality of segmentation representations 510 to generate a representation 622 of a segment 628 (e.g., the first segment of the ocular segmentation profile 520) corresponding to the first ocular zone. In some examples, the set of supplemental segmentation representations 616 may comprise (i) a region of interest segmentation 618 (corresponding to the first region of interest between the first ocular zone and the second ocular zone) generated using the trained region of interest model 460 and/or (ii) one or more other segmentation representations. In some examples, the profile generation module 512 may use the binary segmentation representation 610 as a primary source of information for generating the segment 628 and/or may use the set of supplemental segmentation representations 616 as backup sources of information for generating one or more portions, of the segment 628, where the binary segmentation representation 610 is not sufficiently clear in identifying the first ocular zone. In an example, the profile generation module 512 may identify gaps 612 and/or 614 in the binary segmentation representation 610. In some examples, the gaps 612 and/or 614 may correspond to portions, of the first ocular zone, that the trained single-line model 420 was unable to capture in the binary segmentation representation 610. In response to identifying the gaps 612 and/or 614, the profile generation module 512 may attempt to use one or more of the set of supplemental segmentation representations 616 to augment the binary segmentation representation 610 (e.g., by filling in the gaps 612 and/or 614) to generate the segment 628. For example, one or more first portions 630, 634, 638 and/or 642 of the segment 628 may be generated according to the binary segmentation representation 610. One or more second portions 632, 636 and/or 640 of the segment 628 may be generated according to one or more segmentation representations of the set of supplemental segmentation representations 616, such as the region of interest segmentation 618. For example, the region of interest segmentation 618 may be used to fill in the gaps 612 and/or 614 to produce the segment 628 (and thus the segment 628 may be a more complete representation of the first ocular zone in comparison with the binary segmentation representation 610). Alternatively and/or additionally, the profile generation module 512 may extrapolate and/or interpolate the one or more second portions 632, 636 and/or 640 of the segment 628 based upon the set of supplemental segmentation representations 616, such as the region of interest segmentation 618. In an example, the profile generation module 512 may extrapolate one or more portions of the region of interest segmentation 618 (e.g., one or more portions of a boundary of the region of interest segmentation 618 that is proximal the first ocular zone) to the gaps 612 and/or 614 to generate the segment 628. Embodiments are contemplated in which one or more other types of segmentation representations (e.g., two-line segmentations, such as the second segmentation representation 516, generated using the trained two-line model 440) other than the region of interest segmentation 618 are included in the set of supplemental segmentation representations 616 and/or are used to generate the segment 628 corresponding to the first ocular zone. Thus, in scenarios in which the trained single-line model 420 is unable to capture one or more portions of the first ocular zone (e.g., portions corresponding to the gaps 612 and/or 614), the present disclosure uses segmentation representations generated using a combination of multiple segmentation models to produce a more accurate representation (e.g., the segment 628) of the first ocular zone (e.g., the segment 628 is indicative of where the first ocular zone resides in the gaps 612 and/or 614).

FIG. 6C illustrates an example scenario 650 associated with interactions between the trained single-line model 420 and the trained region of interest model 460 to produce segments (e.g., line segmentations) corresponding to ocular zones including ILM, BM, EZ, RPE and ONL. A first row 652 includes OCT B-scans (from the set of images 502, for example). A second row 654 includes resulting segments produced by the segmentation module 506 (using the trained single-line model 420, for example) based upon respective OCT B-scans of the first row. A third row 656 includes representations of region of interest segmentations (produced using the trained region of interest model 460, for example) for various regions of interest, such as an ILM region of interest segmentation (corresponding to a region of interest between ILM and BM, for example), a BM region of interest segmentation (corresponding to a region of interest between ILM and BM, for example), an EZ region of interest segmentation (corresponding to a region of interest between ILM and EZ, for example), an RPE region of interest segmentation (corresponding to a region of interest between ILM and RPE, for example), and/or an ONL region of interest segmentation (corresponding to a region of interest between ILM and ONL, for example). The representations of the third row 656 provide indications of supplemental ROI portions 658 (shown with a different shading and/or color in FIG. 6C) of the region of interest segmentations that are used as supplemental information by the segmentation module 506 to generate corresponding segments of the second row 654. For example, the segmentation module 506 may extrapolate information from the supplemental ROI portions 658 to generate portions 660 of the segments of the second row 654. In an example shown in FIG. 6C, the segmentation module 506 may (i) generate one or more portions of the EZ line segmentation (in the second row) based upon one or more portions of the EZ region of interest segmentation (in the third row), (ii) generate one or more portions of the RPE line segmentation (in the second row) based upon one or more portions of the RPE region of interest segmentation (in the third row), and/or (iii) generate one or more portions of the ONL line segmentation (in the second row) based upon one or more portions of the ONL region of interest segmentation (in the third row).

In some examples, a plurality of versions of the first segment are generated using various machine learning models and/or combinations of machine learning models. For example, the plurality of versions of the first segment may comprise (i) a first segment version that is generated based upon the first segmentation representation 514 and/or one or more other segmentation representations generated by the trained single-line model 420, (ii) a second segment version that is generated based upon the second segmentation representation 516 and/or one or more other segmentation representations generated by the trained two-line model 440 (e.g., the second segment version may be generated based upon the representation of the first ocular zone in the second segmentation representation 516), (iii) a third segment version that is generated based upon the third segmentation representation 516 and/or one or more other segmentation representations generated by the trained region of interest model 460 (e.g., the third segment version may be generated based upon a boundary, of the representation of the first region of interest in the third segmentation representation 518, that is proximal the first ocular zone), (iv) a fourth segment version that is generated based upon segmentation representations generated by the trained single-line model 420 and the trained region of interest model 460 (e.g., the fourth segment version may be generated based upon the first segmentation representation 514 and the third segmentation representation 518, such as by extrapolating information from the third segmentation representation 518 and/or the first segmentation representation 514 to generate a more complete representation of the first ocular zone), (v) a fifth segment version that is generated based upon segmentation representations generated by the trained two-line model 440 and the trained region of interest model 460 (e.g., the fifth segment version may be generated based upon the second segmentation representation 516 and the third segmentation representation 518, such as by extrapolating information from the second segmentation representation 516 and/or the first segmentation representation 514 to generate a more complete representation of the first ocular zone), (vi) a sixth segment version that is generated based upon segmentation representations generated by the trained single-line model 420, the trained two-line model 440 and the trained region of interest model 460 (e.g., the fifth segment version may be generated based upon the first segmentation representation 514, the second segmentation representation 516 and the third segmentation representation 518, such as by extrapolating information from the first segmentation representation 514, the second segmentation representation 516 and/or the first segmentation representation 514 to generate a more complete representation of the first ocular zone), and/or (vii) one or more other segment versions generated using one or more other combinations of machine learning models and/or segmentation representations. In some examples, the profile generation module 512 may determine segmentation quality scores associated with the plurality of versions of the first segment (e.g., by comparing the plurality of versions with the set of pathological features 526 and/or using other segmentation evaluation techniques) and/or may rank the plurality of versions based upon the segmentation quality scores. The profile generation module 512 may select a top-ranked version of the first segment from the plurality of versions, and/or may include the top-ranked version of the first segment in the ocular segmentation profile 520. Alternatively and/or additionally, the profile generation module 512 may combine two or more versions of the plurality of versions to generate an updated version of the first segment to include in the ocular segmentation profile 520.

In some examples, the plurality of machine learning models 508 may comprise one or more models configured for any number of segmentation targets to generate any number of segments corresponding to any number of ocular zones of the eye. For example, the plurality of machine learning models 508 may comprise (i) a segmentation model (e.g., a three-line model) that is trained to use an image of the set of images 502 to generate a three-zone segmentation representation (for inclusion in the plurality of segmentation representations 510, for example) that is indicative of three segments corresponding to three ocular zones (e.g., three different ocular zones selected from at least one of an ILM, a BM, a RPE, an ONL, an EZ, POS, ELM, an OPL, an INL, an IPL, a GCL, a RNFL, etc.) comprising the first ocular zone and two other ocular zones, (ii) a segmentation model (e.g., a four-line model) that is trained to use an image of the set of images 502 to generate a four-zone segmentation representation (for inclusion in the plurality of segmentation representations 510, for example) that is indicative of four segments corresponding to four ocular zones (e.g., four different ocular zones selected from at least one of an ILM, a BM, a RPE, an ONL, an EZ, POS, ELM, an OPL, an INL, an IPL, a GCL, a RNFL, etc.) comprising the first ocular zone and three other ocular zones, and/or one or more other segmentation models associated with one or more other numbers of segmentation targets and/or ocular zones. In some examples, at least one of the three-zone segmentation representation, the four-zone segmentation representation, etc. may be used by the profile generation module 512 (with other segmentation representations of the plurality of segmentation representations 510, for example) to generate the first segment corresponding to the first ocular zone (and/or one or more other segments of the ocular segmentation profile 520).

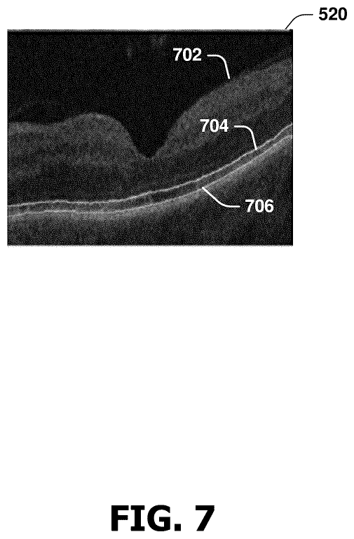

FIG. 7 illustrates an example representation of the ocular segmentation profile 520. In FIG. 7, the plurality of segments of the ocular segmentation profile 520 may overlay an image (e.g., an OCT B-scan of at least a portion of the eye of the person) of the set of images 502. The plurality of segments of the ocular segmentation profile 520 may comprise an ILM segment 702 corresponding to an ILM of the eye, an EZ band segment 704 corresponding to an EZ band of the eye and/or an RPE segment 706 corresponding to a RPE of the eye. In an example, the first ocular zone may correspond to the ILM represented by the ILM segment 702, the second ocular zone may correspond to the EZ band represented by the EZ band segment 704, and/or the plurality of ocular zones may comprise a third ocular zone corresponding to the RPE represented by the RPE segment 706. In an example in which the first ocular zone corresponds to the ILM, the ILM segment 702 (e.g., the first segment) may be generated based upon segmentation representations (e.g., segmentation representations 514, 516, 518, 606, 610 and/or 618) generated using machine learning models (e.g., the trained single-line model 420, the trained two-line model 440 and/or the trained region of interest model 460) trained on training information (e.g., the first plurality of images 402, the first label information 410, the second plurality of images 422, the second label information 430, the third plurality of images 442 and/or the third label information 450) identifying segments corresponding to ILM. In an example in which the third ocular zone corresponds to the RPE, the RPE segment 706 of the ocular segmentation profile 520 may be generated based upon segmentation representations generated using machine learning models (e.g., a second trained single-line model trained using one or more of the techniques provided herein with respect to the trained single-line model 420, a second trained two-line model trained using one or more of the techniques provided herein with respect to the trained two-line model 440 and/or a second trained region of interest model trained using one or more of the techniques provided herein with respect to the trained region of interest model 460) trained on training information identifying segments corresponding to RPE, such as using one or more of the techniques provided herein with respect to generating the plurality of segmentation representations 510 and/or generating the first segment based upon the plurality of segmentation representations 510.

In some examples, the feature extraction module 522 is configured to perform one or more feature extraction tasks on one or more images of the set of images 502 (e.g., one or more en face images and/or B-scans) and/or one or more segmentation representations of the plurality of segmentation representations 510 to identify the set of pathological features 526. In some examples, the feature extraction module 522 may comprise a plurality of feature extraction models for detecting different pathological feature types. For example, the one or more feature extraction tasks may comprise (i) the first feature extraction task performed using the first trained feature extraction model 480 for detecting the one or more first pathological feature types, (ii) a second feature extraction task performed using a second trained feature extraction model 524 (e.g., a model, of the feature extraction module 522, trained using one or more of the techniques provided herein with respect to the first trained feature extraction model 480) for detecting one or more second pathological feature types (e.g., at least one of IRF, SRF, SRMat, SHRM, GA, drusen, inflammatory debris, inflammatory lesion, hypertransmission defect, hypotransmission defect, cystic fluid, general fluid, sub-RPE fluid, lesion, etc.), (iii) a third feature extraction task performed using a third trained feature extraction model (e.g., a model, of the feature extraction module 522, trained using one or more of the techniques provided herein with respect to the first trained feature extraction model 480) for detecting one or more third pathological feature types (e.g., at least one of IRF, SRF, SRMat, SHRM, GA, drusen, inflammatory debris, inflammatory lesion, hypertransmission defect, hypotransmission defect, cystic fluid, general fluid, sub-RPE fluid, lesion, etc.), and/or (iv) one or more other feature extraction tasks performed using one or more other models of the feature extraction module 522 for detecting other pathological feature types.

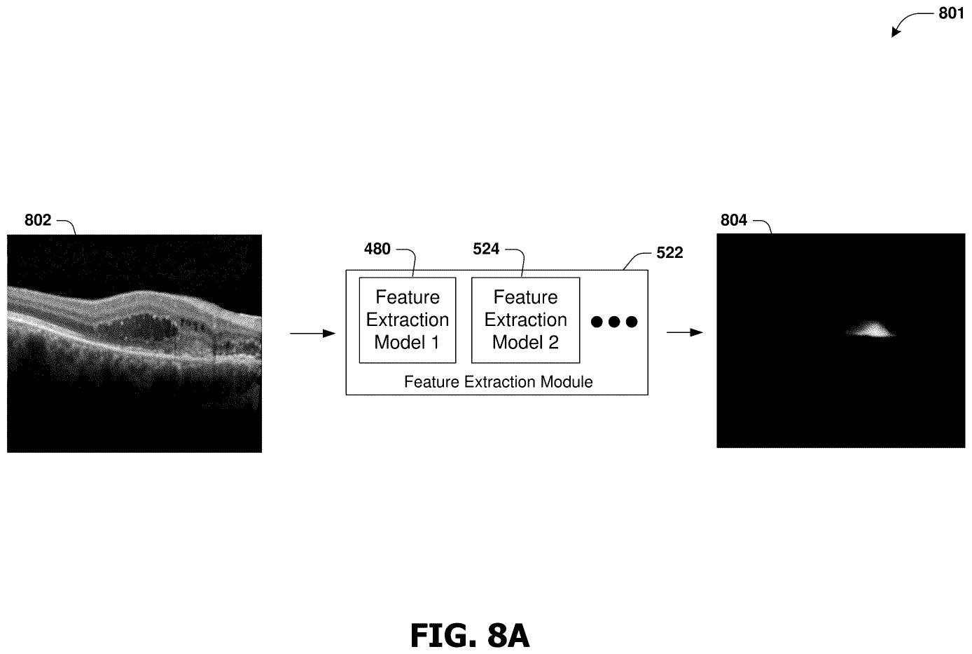

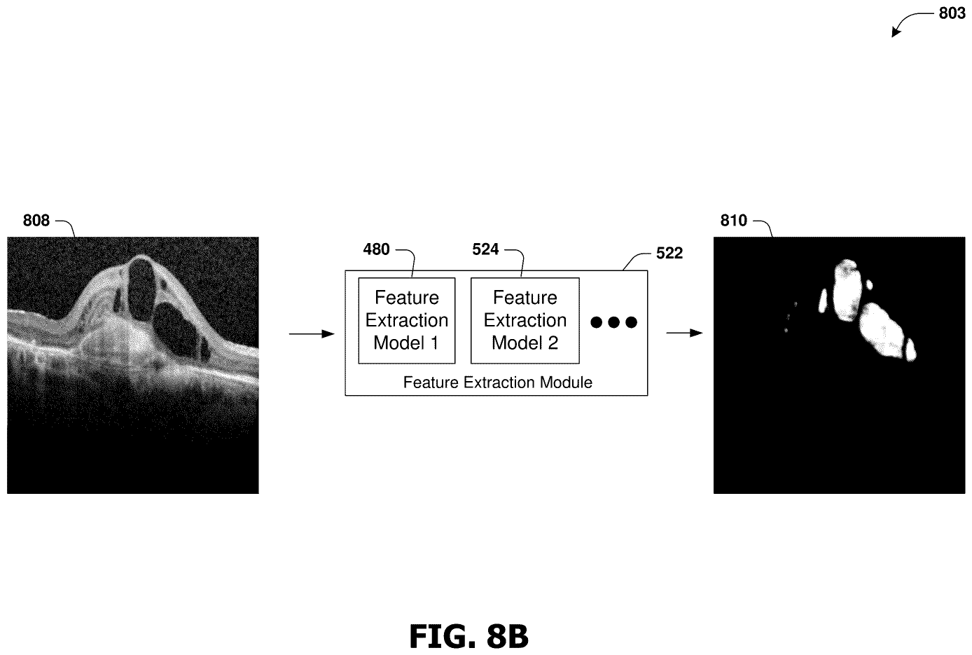

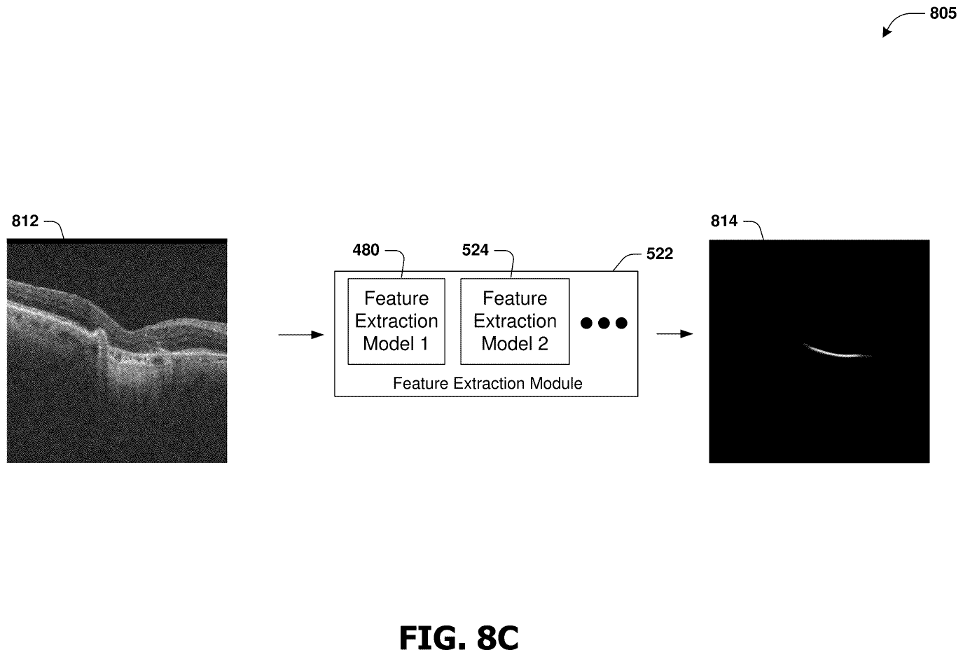

FIGS. 8A-8C illustrate example scenarios associated with determining the set of pathological features 526. FIG. 8A illustrates a first scenario 801 in which an image 802 (e.g., an OCT B-scan of at least a portion of the eye of the person) of the set of images 502 is processed using the feature extraction module 522 to generate a feature representation 804 corresponding to subretinal material (SRMat). For example, the feature representation 804 may be indicative of a portion, of the eye, where the feature extraction module 522 detected SRMat in the image 802. Thus, the set of pathological features 526 may comprise SRMat represented by the feature representation 804. The feature representation 804 may be generated using one or more models (e.g., the first trained feature extraction model 480), of the plurality of feature extraction models, that are trained on training information identifying segments corresponding to SRMat.

FIG. 8B illustrates a second scenario 803 in which an image 808 (e.g., an OCT B-scan of at least a portion of the eye of the person) of the set of images 502 is processed using the feature extraction module 522 to generate a feature representation 810 corresponding to fluid (e.g., at least one of cystic fluid, general fluid, sub-RPE fluid, etc.). For example, the feature representation 810 may be indicative of a portion, of the eye, where the feature extraction module 522 detected fluid (e.g., at least one of cystic fluid, general fluid, sub-RPE fluid, etc.) in the image 802. Thus, the set of pathological features 526 may comprise fluid (e.g., at least one of cystic fluid, general fluid, sub-RPE fluid, etc.) represented by the feature representation 810. The feature representation 810 may be generated using one or more models (e.g., the first trained feature extraction model 480), of the plurality of feature extraction models, that are trained on training information identifying segments corresponding to fluid (e.g., at least one of cystic fluid, general fluid, sub-RPE fluid, etc.).

FIG. 8C illustrates a third scenario 805 in which an image 812 (e.g., an OCT B-scan of at least a portion of the eye of the person) of the set of images 502 is processed using the feature extraction module 522 to generate a feature representation 814 corresponding to GA hypertransmission. For example, the feature representation 814 may be indicative of a portion, of the eye, where the feature extraction module 522 detected GA hypertransmission in the image 812. Thus, the set of pathological features 526 may comprise GA hypertransmission represented by the feature representation 814. The feature representation 814 may be generated using one or more models (e.g., the first trained feature extraction model 480), of the plurality of feature extraction models, that are trained on training information identifying segments corresponding to GA hypertransmission.

In some examples, the feature extraction module 522 is configured to generate (and/or update and/or adjust) a feature representation (e.g., feature representations 804, 810 and/or 814) identifying a feature of the set of pathological features 526 based upon one or more segmentation representations of the plurality of segmentation representations 510 (and/or the ocular segmentation profile 520). Accordingly, the set of pathological features 526 may provide feature representations that are more accurate representations of pathological features of the eye by way of taking detected ocular zones into account. For example, the feature extraction module 522 may compare a position of SRMat (represented by the feature representation 804, for example) with a position of a RPE (e.g., an RPE segment indicated by one or more segmentation representations of the plurality of segmentation representations 510 and/or the ocular segmentation profile 520). In response to determining that at least a portion of the SRMat is below the RPE (which is anatomically not possible, for example), the feature extraction module 522 may delete at least the portion of the SRMat (and/or may delete an entirety of the SRMat) and/or may refine a representation of the SRMat based upon the RPE segment.

Alternatively and/or additionally, the feature extraction module 522 may adjust an area of GA (represented by the feature representation 814, for example) based upon interrogation of relative BM/RPE distances (which may determined by calculating distances between an RPE segment and a BM segment indicated by one or more segmentation representations of the plurality of segmentation representations 510 and/or the ocular segmentation profile 520, for example). For example, in response to identifying a region in which one or more distances between the BM and the RPE are less than a threshold distance, at least a portion of the RPE segment and/or at least a portion of the BM segment (e.g., respective portions of the RPE segment and/or the BM segment that are within the region, for example) may be collapsed into the area of GA. Alternatively and/or additionally, in response to identifying a region in which one or more distances between the BM and the RPE are greater than the threshold distance, the feature extraction module 522 may delete at least a portion of the area of GA.

Presence of a pathological feature in a region of the eye of the person may impact ocular zone segmentation associated with the region. In some examples, the profile generation module 512 of the segmentation module 506 is configured to generate the ocular segmentation profile 520 based upon the set of pathological features 526 output by the feature extraction module 522. Accordingly, the ocular segmentation profile 520 may provide segments that are more accurate representations of ocular zones of the eye by way of taking detected pathological features into account when generating the ocular segmentation profile 520.

The profile generation module 512 may generate a first version of the first segment corresponding to the first ocular zone based upon one, some or all segmentation representations of the plurality of segmentation representations 510. The profile generation module 512 may compare one or more positions of one or more respective features of the set of pathological features 526 with a position of the first segment, and/or may generate a second version (e.g., an updated version) of the first segment based upon the comparison (e.g., the profile generation module 512 may modify the first version of the first segment based upon the comparison to generate the second version of the first segment). The profile generation module 512 may generate the ocular segmentation profile 520 to comprise the second version of the first segment (rather than the first version of the first segment, for example).

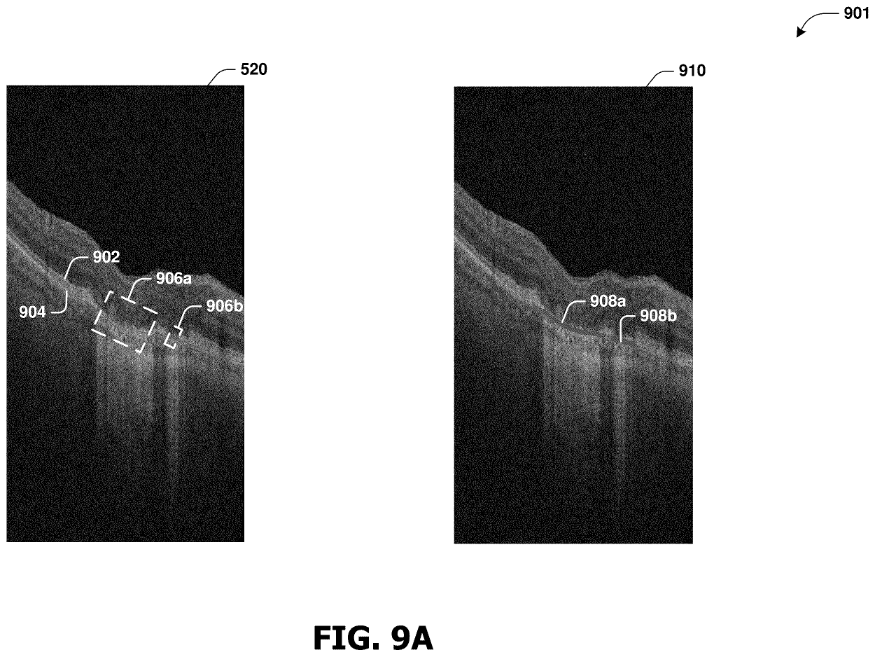

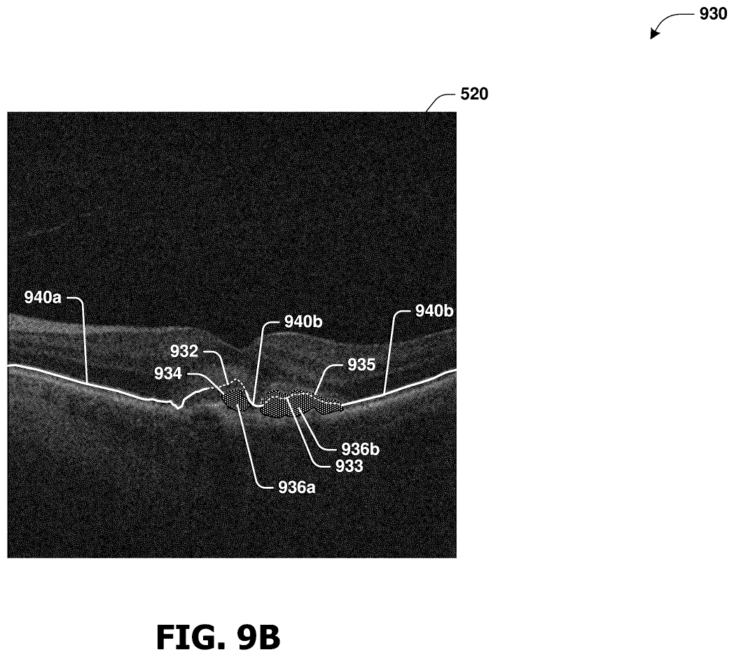

FIG. 9A illustrates an example interaction between the ocular segmentation profile 520 and the set of pathological features 526 in an example scenario 901. In the example scenario 901, the ocular segmentation profile 520 may comprise an EZ band segment 902 corresponding to an EZ band of the eye and/or a Bruch's membrane (BM) segment 904 corresponding to a BM of the eye. The set of pathological features 526 may comprise GA (e.g., GA hypertransmission) represented by a feature representation 910 indicative of GA feature segments 908a and/or 908b corresponding to portions, of the eye, where the feature extraction module 522 detected GA (e.g., GA hypertransmission). In FIG. 9A, the EZ band segment 902, the BM segment 904 and/or the GA feature segments 908a and/or 908b may overlay a (same) image (e.g., an OCT B-scan of at least a portion of the eye of the person) of the set of images 502. In some examples, the profile generation module 512 may identify one or more GA-impacted portions, of the EZ band segment 902, that are proximal respective positions of the GA feature segments 908a and/or 908b. For example, a GA-impacted portion may correspond to a portion, of the EZ band segment 902, that is within a threshold distance of a GA feature segment of the GA feature segments 908a and/or 908b. Alternatively and/or additionally, the profile generation module 512 may define GA-impacted regions 906a and/or 906b relative to the EZ band segment 902 based upon one or more positions of the GA feature segments 908a and/or 908b, and/or may identify a GA-impacted portion (of the one or more GA-impacted portions) as a portion, of the EZ band segment 902, that is within the GA-impacted regions 906a and/or 906b. In some examples, the profile generation module 512 generates the one or more GA-impacted portions of the EZ band segment 902 based upon the BM segment 904. For example, the profile generation module 512 may collapse at least some of a GA-impacted portion of the EZ band segment 902 into at least a portion of the BM segment 904 (such that a portion or an entirety of the one or more GA-impacted portions of the EZ band segment 902 is aligned with, overlapping with, concurrent with and/or identical to at least a portion of the BM segment 904, for example). In an example, within the GA-impacted regions 906a and/or 906b (defined based upon the GA feature segments 908a and/or 908b), the profile generation module 512 may generate the EZ band segment 902 to be equal to the BM segment 904 (such that the EZ band segment 902 and the BM segment 904 are represented by a single line in GA-impacted regions 906a and/or 906b, for example). In some examples, in response to identifying the GA feature segments 908a and/or 908b, the profile generation module 512 may use the BM segment 904 to modify the one or more GA-impacted portions of a prior version of the EZ band segment 902 (e.g., the prior version of the EZ band segment 902 may be generated based upon one, some or all segmentation representations of the plurality of segmentation representations 510) to generate an updated version of the EZ band segment 902 (e.g., the one or more GA-impacted portions of the prior version of the EZ band segment 902 may be collapsed into the BM segment 904 to generate the updated version of the EZ band segment 902 shown in FIG. 9A). Alternatively and/or additionally, the segmentation module 506 may determine one or more characteristics of the EZ band segment 902 and/or the EZ band based upon the GA feature segments 908a and/or 908b. For example, the segmentation module 506 may label the GA-impacted portion of the EZ band segment 902 as having a first thickness (e.g., a thickness of 0).

Alternatively and/or additionally, the segmentation module 506 may determine one or more characteristics of the BM segment 904 and/or the BM based upon the GA feature segments 908a and/or 908b. A GA-impacted portion of the BM segment 904 may be determined (e.g., the GA-impacted portion of the BM segment 904 may comprise a portion, of the BM segment 904, that is within a threshold distance of the GA feature segments 908a and/or 908b and/or a portion, of the BM segment 904, that is within one of the GA-impacted regions 906a and/or 906b). For example, the segmentation module 506 may label the GA-impacted portion of the BM segment 904 as having a second thickness (e.g., a thickness of 0), which may be the same as or different than the first thickness.

In some examples, one or more other types of segments (other than the EZ band segment 902) may be generated based upon the GA feature segments 908a and/or 908b and/or other types of features segments (associated with other pathological feature types, for example). In an example, the ocular segmentation profile 520 may comprise an RPE segment corresponding to an RPE of the eye. A GA-impacted portion of the RPE segment may be determined and/or the GA-impacted portion of the RPE segment may be generated based upon the BM segment 904 (and/or other segment) using one or more of the techniques provided herein with respect to the EZ band segment 902. For example, the profile generation module 512 may collapse at least some of a GA-impacted portion of the RPE segment into at least a portion of the BM segment 904 (such that a portion or an entirety of one or more GA-impacted portions of the RPE segment is aligned with, overlapping with, concurrent with and/or identical to at least a portion of the BM segment 904, for example). Alternatively and/or additionally, in an area where GA is not identified (e.g., an area outside of the GA-impacted regions 906a and/or 906b), the profile generation module 512 may move the RPE segment one or more pixels (e.g., one pixel) anterior to the BM segment 904 (to mitigate and/or eliminate false positive GA and/or small speckle noise, for example). Alternatively and/or additionally, the segmentation module 506 may label the GA-impacted portion of the RPE segment as having a third thickness (e.g., a thickness of 0), which may be the same as and/or different than the first thickness and/or the second thickness.

FIG. 9B illustrates an example interaction between the ocular segmentation profile 520 and the set of pathological features 526 in an example scenario 930. In the example scenario 930, an EZ band segment of the ocular segmentation profile 520 is modified from a first version (represented by solid white line and dashed white line) to a second version (represented by the solid white line and dotted white line) based upon the set of pathological features 526. For example, the set of pathological features 526 may comprise Subretinal material (SRMat) represented by SRMat feature segments 936a and/or 936b (e.g., SRMat feature segments 936a and/or 936b may correspond to portions, of the eye, where the feature extraction module 522 detected SRMat). In some examples, the EZ band segment is modified from the first version to the second version by replacing one or more sections shown with dashed white line with one or more sections shown with dotted white line based upon SRMat feature segments 936a and/or 936b. For example, the modification may include replacing at least some of portion 932 (which overlies the SRMat feature segment 936a, for example) of the first version with portion 934 (which is aligned with a top surface of the SRMat feature segment 936a, for example) of the second version based upon the SRMat feature segment 936a. Alternatively and/or additionally, the modification may include replacing at least some of portion 933 (which overlaps with the SRMat feature segment 936b, for example) of the first version with portion 935 (which is aligned with a top surface of the SRMat feature segment 936b, for example) of the second version based upon the SRMat feature segment 936b. Accordingly, the profile generation module 512 may generate the second version of the EZ band segment to account for the SRMat feature segments 936a and/or 936b, and/or may include the second version of the EZ band segment in the ocular segmentation profile 520. In some examples, portions 940a, 940b and/or 940c of the EZ band segment (shown with solid white line in FIG. 9B) are common to (and/or unchanged between) the first version and the second version of the EZ band segment.

In an example in which the set of pathological features 526 detected by the feature extraction module 522 is indicative of subretinal fluid (SRF), the segmentation module 506 may generate (and/or update and/or modify) one or more segments of the ocular segmentation profile 520 based upon a position of the SRF. For example, in response to identifying a region in which an EZ band segment overlaps with or is below the SRF, the feature extraction module 522 may modify the EZ band segment, such as by moving the EZ band segment to an elevation above the SRF.

In some examples, one or more segmentation models (e.g., the trained single-line model 420, the trained two-line model 440, the trained region of interest model 460, one or more other machine learning models of the plurality of machine learning models 508, etc.) of the segmentation module 506 may be updated (e.g., further trained) using feedback information derived from the set of pathological features 526. For example, the feedback information may be indicative of (i) a modification made to update a segment (e.g., modify the first segment from the first version of the first segment to the second version of the first segment) based upon a feature of the set of pathological features 526, (ii) a difference between the first version of the first segment and the second version of the first segment, and/or (iii) a correct version of the first segment (e.g., the second version of the first segment). It may be appreciated that updating and/or training the one or more segmentation models based upon the feedback information may create a closed-loop process allowing results of feature extraction and/or interactions between feature extraction and segmentation as feedback to tailor settings of the one or more segmentation models of the segmentation module 506. Closed-loop control may reduce errors and produce more efficient operation of a computer system which implements the segmentation module 506. The reduction of errors and/or the efficient operation of the computer system may improve operational stability and/or predictability of operation. Accordingly, using processing circuitry to implement closed loop control described herein may improve operation of underlying hardware of the computer system.

In some examples, the system 501 comprises an ocular evaluation module 528 is used to analyze the ocular segmentation profile 520 and/or the set of pathological features 526 to determine a set of (one or more) parameters. The set of parameters may be usable for diagnosing and/or treating the person. In an example, the set of parameters may comprise (i) ellipsoid zone (EZ) integrity, (ii) a measure (e.g., volume) of intraretinal fluid (IRF) (e.g., longitudinal IRF volume), (ii) a measure (e.g., volume) of subretinal fluid (SRF) (e.g., longitudinal SRF volume), (iii) a measure (e.g., volume) of subretinal hyperreflective material (SHRM) and/or a measure (e.g., volume) of subretinal material (SRMat), (iv) a measure (e.g., volume and/or thickness) of retinal pigment epithelium (RPE), (v) a measure (e.g., volume and/or thickness) of geographic atrophy (GA), (vi) a measure of retinal toxicity (e.g., hydroxychloroquine toxicity and/or chloroquine toxicity in the person's eye), (vii) a correlation between best corrected visual acuity (BCVA) with one or more other parameters and/or features, and/or (viii) one or more other measures associated with one or more features of the set of pathological features 526 and/or one or more segments (e.g., ocular zone segments) of the ocular segmentation profile 520.

In some examples, the ocular evaluation module 528 may analyze the set of parameters, the ocular segmentation profile 520 and/or the set of pathological features 526 to (i) diagnose the person with a first eye condition (e.g., one or more retinal diseases), (ii) determine a progression and/or severity level of the first eye condition, (iii) determine a risk level of the first eye condition and/or (iv) generate a treatment plan for the person (e.g., a treatment plan for treating the first eye condition). In some examples, a treatment of the person may be controlled based upon the treatment plan. In an example, the first eye condition may comprise at least one of macular degeneration, age-related macular degeneration (AMD) (e.g., wet AMD and/or dry AMD), diabetic retinopathy, one or more inherited retinal diseases, one or more atrophic eye diseases (e.g., at least one of retinitis pigmentosa, Stargardt disease, etc.), inflammatory eye disease, retinal toxicity (e.g., hydroxychloroquine toxicity and/or chloroquine toxicity), and/or one or more other eye conditions.

In some examples, the ocular evaluation module 528 may generate an ocular report 530 based upon the set of parameters, the ocular segmentation profile 520, the set of pathological features 526, the first eye condition, the risk level, the progression and/or severity level and/or the treatment plan. For example, the ocular report 530 may comprise one or more graphical objects (e.g., one or more charts, one or more B-scans, etc.) and/or text indicative of the first eye condition, the risk level, the progression and/or severity level, the treatment plan, one or more segments of the ocular segmentation profile 520 and/or one or more features of the set of pathological features 526. In an example, the ocular report 530 may comprise a representation of the ocular segmentation profile 520, which may comprise one or more ocular zone segments (of the plurality of segments) overlaid onto an image, such as a B-scan, of the eye of the person (e.g., the ocular report 530 may comprise the example representation of the ocular segmentation profile 520 shown in FIG. 7).

In some examples, the ocular report 530 may be transmitted to a client device (e.g., at least one of a phone, a tablet, a laptop, a computer, a wearable device, a smart device, a television, any other type of computing device, hardware and/or software) and/or displayed via a graphical user interface. For example, the ocular report 530 may be displayed to one or more healthcare professionals (e.g., physician, surgeon, nurse, etc.) that are associated with the treatment of the person. For example, the ocular report 530 may provide a healthcare professional with information about the person's response to the treatment (e.g., whether the first eye condition of the person is improving or worsening) that can enable the healthcare professional to have an improved understanding of an effectiveness of the treatment thus far and/or make a more informed decision of one or more next steps of the treatment. Alternatively and/or additionally, the ocular report 530 may enable the one or more healthcare professionals to detect the first eye condition of the person more quickly and/or sooner, which may allow the one or more healthcare professionals to begin treating the first eye condition at an earlier stage such that the treatment is more effective (than if the first eye condition was detected and/or treated at a later stage, for example).

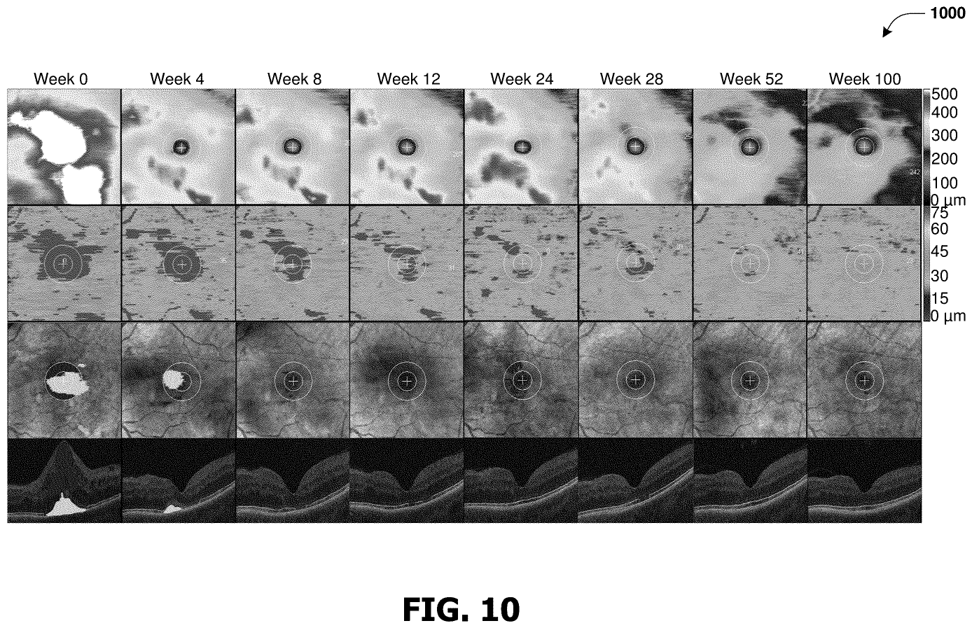

In some examples, the ocular report 530 may be indicative of historical information associated with the person and/or the first eye condition comprising (i) one or more historical ocular zone segments, pathological features and/or parameters detected and/or measured using the segmentation module 506, the feature extraction module 522 and/or the ocular evaluation module 528, (ii) changes in ocular zone segments, pathological features and/or parameters over time and/or (iii) comparisons between ocular zone segments, pathological features and/or parameters associated with different times. For example, FIG. 10 illustrates an example representation 1000 of at least a portion of the ocular report 530. The example representation 1000 may be associated with a representative case with diabetic macular edema (DME) treated with intravitreal aflibercept injection (IAI) demonstrating a longitudinal change in en face retinal thickness mapping, ellipsoid zone (EZ) mapping, intraretinal fluid (IRF)/subretinal fluid (SRF) mapping, and horizontal OCT B-scan image. Among 10 follow-up time points that have been evaluated in this subanalysis, 8 selected time points are shown in the example representation 1000, including the baseline (farthest left column) and weeks 4, 8, 12, 24 28, 52, and 100 (farthest right column). An inner circle represents the macular radius of 0.5 mm (corresponds to central subfield), and an outer circle represents the macular radius of 1.0 mm (corresponds to central macula) in the en face macular map. The top row of the example representation 1000 represents en face retinal thickness mapping. The second from top row of the example representation 1000 represents en face EZ mapping representing the topographical thickness between EZ and retinal pigment epithelium (RPE). Ellipsoid zone RPE attenuation mainly localized within the central macular area gradually decreased in size through week 52. The third from top row of the example representation 1000 represents IRF and SRF mapping, which may be indicative of IRF within central macula and/or SRF. The bottom row of the example representation 1000 represents horizontal B-scan crossing the central fovea displaying segmented retinal layer boundaries and visually discernable fluid. In the example representation 1000, segments (e.g., segmentation lines and/or region of interest segmentations) corresponding to internal limiting membrane (ILM), EZ band, RPE, IRF, and SRF may be marked by areas with different colors and/or shading.

In some examples, the system 501 comprises a segmentation evaluation module 532 for determining a segmentation quality metric associated with the ocular segmentation profile 520 based upon the set of pathological features 526. For example, the segmentation evaluation module 532 may compare one or more positions of one or more respective features of the set of pathological features 526 with one or more positions of one or more segments of the ocular segmentation profile 520, and/or may determine the segmentation quality metric based upon the comparison. In some examples, in response to determining that the segmentation quality metric does not meet (e.g., does not exceed) a threshold segmentation quality, the segmentation module 506 may perform one or more acts to produce a new and/or updated ocular segmentation profile. The one or more acts may comprise requesting one or more new and/or updated images (e.g., OCT images, such as OCT B-scans and/or A-scans) of the eye of the person that may be used to produce a higher quality segmentation profile. Alternatively and/or additionally, the one or more acts may comprise adjusting one or more settings of the segmentation module 506, and/or generating a new and/or updated ocular segmentation profile using the adjusted settings.

The person having the first eye condition may impact ocular zone segmentation associated with the region. In some examples, the profile generation module 512 of the segmentation module 506 is configured to generate the ocular segmentation profile 520 based upon set of parameters and/or the first eye condition. Accordingly, the ocular segmentation profile 520 may provide segments that are more accurate representations of ocular zones of the eye by way of taking the set of parameters and/or the first eye condition into account when generating the ocular segmentation profile 520.

In some examples, one or more settings used by the segmentation module 506 to generate the ocular segmentation profile 520 may be adjusted and/or tuned based upon the set of parameters and/or the first eye condition. For example, the one or more settings may be set according to a first configuration associated with the first eye condition. The first configuration may be selected from a plurality of configurations associated with various eye conditions and/or various parameters (e.g., the plurality of configurations may comprise at least one of a diabetic retinopathy configuration that is used for persons diagnosed with and/or at risk of diabetic retinopathy, a wet AMD configuration that is used for persons diagnosed with and/or at risk of wet AMD, etc.). The one or more settings may correspond to model parameters of one, some or all machine learning models of the plurality of machine learning models 508.

In an example scenario, the first ocular zone may correspond to an internal limiting membrane (ILM) of the eye and/or the second ocular zone may correspond to a Bruch's membrane (BM) of the eye. The trained single-line model 420 may analyze an image of the set of images 502 (e.g., an OCT B-scan of at least a portion of the eye of the person) to generate a single-line representation (e.g., the first segmentation representation 514) comprising a segment (e.g., a line segmentation) corresponding to the ILM. The trained two-line model 440 may analyze the image to generate a two-line representation (e.g., the second segmentation representation 516) comprising a segment (e.g., a line segmentation) corresponding to the ILM and a segment (e.g., a line segmentation) corresponding to the BM. The trained region of interest model 460 may analyze the image to generate a region of interest representation (e.g., the third segmentation representation 518) comprising a segment (e.g., a region of interest segmentation) corresponding to the first region of interest between the ILM and the BM. The segmentation module 506 may use the single-line representation, the two-line representation and/or the region of interest representation to determine the first segment, of the ocular segmentation profile 520, corresponding to the ILM, thereby providing for increased accuracy of the first segment as a result of using multiple models that consider different contexts in different ways (e.g., the trained single-line model 420 considers local context relative to the ILM and/or the trained two-line model 440 and/or the trained region of interest model 460 may consider global context relative to the ILM).

In some examples, each machine learning model of one, some and/or all machine learning models of the present disclosure (e.g., the trained single-line model 420, the trained two-line model 440, the trained region of interest model 460, one or more other machine learning models of the plurality of machine learning models 508, the first trained feature extraction model 480, the second trained feature extraction model 524, one or more other machine learning models of the feature extraction module 522, etc.) may be configured for biomedical image segmentation and/or may comprise at least one of a neural network, such as a convolutional neural network (e.g., a convolutional neural network with a deep learning U-net architecture), a tree-based model, a machine learning model used to perform linear regression, a machine learning model used to perform logistic regression, a decision tree model, a support vector machine (SVM), a Bayesian network model, a k-Nearest Neighbors (k-NN) model, a K-Means model, a random forest model, a machine learning model used to perform dimensional reduction, a machine learning model used to perform gradient boosting, etc.

In some examples, a testing process is performed for each machine learning model of one, some and/or all machine learning models of the present disclosure. The testing process may include (i) using the machine learning model to generate a segmentation representation corresponding to an ocular zone based upon an image (e.g., an OCT image) for which a label (e.g., ground truth information corresponding to actual location of the ocular zone relative to the image) is available, (ii) comparing the first segmentation representation with the label, and/or (iii) updating and/or optimizing the machine learning model (e.g., adjusting one or more tunable settings of the machine learning model) based upon the comparison. For example, a pixel offset between the first segmentation representation and the label may be determined. The pixel offset may correspond to an average deviation of the first segmentation representation from the label. The machine learning model may be updated and/or optimized based upon the pixel offset. In some examples, using the pixel offset to evaluate the segmentation representation and/or update and/or optimize the machine learning model (as compared with some other types of metrics such as area under curve (AUC), F-score, etc., for example) provides for (i) improved segmentation precision, (ii) reduced error rates, and/or (iii) improved future generation of the machine learning model. In some examples, segmentation gaps and/or errors may be penalized (e.g., penalized heavily) by defaulting gap sections to a value (e.g., y=0). In some examples, the trained single-line model 420 may be associated with a pixel offset (e.g., mean offset from ground truth in units of pixels) of about 25.38. The trained two-line model 440 may be associated with a pixel offset (e.g., mean offset from ground truth in units of pixels) of about 2.17. The trained region of interest model 460 may be associated with a pixel offset (e.g., mean offset from ground truth in units of pixels) of about 5.65.

In some examples, deep learning convolutional segmentation implemented by the segmentation module 506 may work by building local texture features into abstract understanding through layered learning. For images in which different things have similar local appearances, incentivizing deep learning models (e.g., the plurality of machine learning models 508) to grasp global context can be useful for emphasizing more meaningful global features. The use of multiple lines (e.g., two line segmentations generated using the trained two-line model 440) bounding an area in question may significantly improve the reliability of the models in high pathology situations. Alternatively and/or additionally, using the trained two-line model 440 to generate the second segmentation representation 516 (which may frame the first region of interest between the first ocular zone and the second ocular zone) may force the trained two-line model 440 (and/or the segmentation module 506) to consider context (e.g., global OCT architecture) indirectly. Alternatively and/or additionally, using the trained region of interest model 460 to generate the third segmentation representation 518 (comprising a segment corresponding to the first region of interest between the first ocular zone and the second ocular zone) may force the trained region of interest model 460 (and/or the segmentation module 506) to consider context (e.g., global context) directly. Alternatively and/or additionally, segmentation representations generated using the trained single-line model 420 may be boundary-sensitive, whereas segmentation representations generated using the trained region of interest model 460 may be boundary-robust. Accordingly, combining capabilities and/or results of multiple machine learning models with different segmentation tasks (e.g., the trained single-line model 420, the trained two-line model 440 and/or the trained region of interest model 460) using the techniques provided herein may provide for improved accuracy of the ocular segmentation profile 520 and/or the ocular report 530.

In some examples, the plurality of machine learning models 508 may comprise a low magnification machine learning model and/or a high magnification machine learning model. The low magnification machine learning model may be trained using images with relatively lower magnification levels and/or the high magnification machine learning model may be trained using images with relatively higher magnification levels. The low magnification machine learning model may incorporate relatively greater context while operating with relatively poorer precision, whereas the high magnification machine learning model may operate at relatively higher precision, but with less contextual information that may lead to mistakes. The low magnification machine learning model and the high magnification machine learning model may be used to generate segmentation representations associated with the first ocular zone based upon the set of images 502. The segmentation representations (e.g., a low magnification segmentation representation and a high magnification segmentation representation) may be used to determine the first segment, of the ocular segmentation profile 520, corresponding to the first ocular zone. It may be appreciated that combining low magnification and high magnification machine learning models may improve overall accuracy and/or local precision of the segmentation module 506.

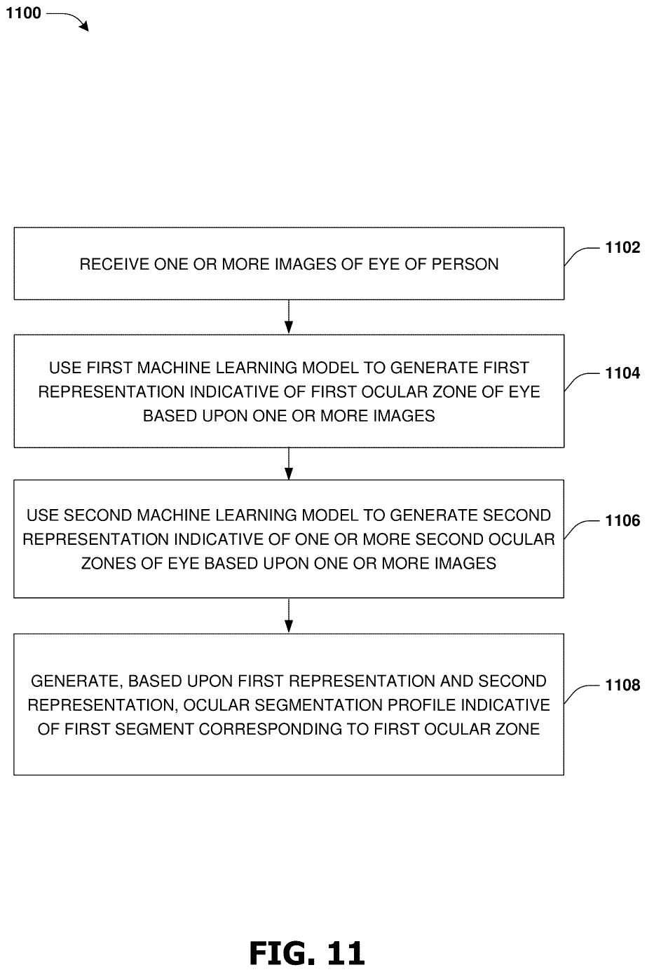

An embodiment of generating an ocular segmentation profile is illustrated by an example method 1100 of FIG. 11. At 1102, one or more images (e.g., the set of images 502) of an eye of a person may be received. At 1104, a first machine learning model (e.g., the trained single-line model 420) may be used to generate a first representation (e.g., the first segmentation representation 514) indicative of a first ocular zone based upon the one or more images. At 1106, a second machine learning model (e.g., the trained two-line model 440 and/or the trained region of interest model) may be used to generate a second representation (e.g., the second segmentation representation 516 and/or the third segmentation representation 518) indicative of one or more second ocular zones of the eye based upon the one or more images. At 1108, an ocular segmentation profile (e.g., the ocular segmentation profile 520) indicative of a first segment corresponding to the first ocular zone of the eye is generated based upon the first representation and/or the second representation. For example, the ocular segmentation profile may be indicative of a plurality of segments corresponding to a plurality of ocular zones of the eye.

According to some embodiments, a computer-implemented method is provided. The computer-implemented method includes receiving one or more images of an eye of a person; using a first machine learning model to generate a first representation indicative of a first ocular zone of the eye based upon the one or more images; using a second machine learning model to generate a second representation indicative of one or more second ocular zones of the eye based upon the one or more images; and generating, based upon the first representation and the second representation, an ocular segmentation profile indicative of a plurality of segments corresponding to a plurality of ocular zones of the eye.

According to some embodiments, the computer-implemented method includes using a third machine learning model to generate a third representation indicative of the one or more second ocular zones of the eye based upon the one or more images, wherein generating the ocular segmentation profile is performed based upon the third representation.

According to some embodiments, the one or more second ocular zones include the first ocular zone and a second ocular zone; the second representation defines a region of interest between the first ocular zone and the second ocular zone; and the third representation includes a representation of the first ocular zone and a representation of the second ocular zone.

According to some embodiments, the computer-implemented method includes identifying a plurality of images associated with a plurality of persons; and training a machine learning model using the plurality of images and label information associated with the plurality of images to generate the second machine learning model, wherein the label information is indicative of a segment, of an image of the plurality of images, that corresponds to the region of interest between the first ocular zone and the second ocular zone.

According to some embodiments, the computer-implemented method includes identifying a plurality of images associated with a plurality of persons; and training a machine learning model using the plurality of images and label information associated with the plurality of images to generate the third machine learning model, wherein the label information is indicative of: a first segment, of an image of the plurality of images, that corresponds to the first ocular zone; and a second segment, of the image, that corresponds to the second ocular zone.

According to some embodiments, the computer-implemented method includes identifying a plurality of images associated with a plurality of persons; and training a machine learning model using the plurality of images and label information associated with the plurality of images to generate the first machine learning model, wherein the label information is indicative of a segment, of an image of the plurality of images, that corresponds to the first ocular zone.

According to some embodiments, the computer-implemented method includes using a feature extraction machine learning model to identify one or more pathological features of the eye based upon the first representation, the second representation and/or the one or more images, wherein generating the ocular segmentation profile is performed based upon the one or more pathological features.

According to some embodiments, generating the ocular segmentation profile includes: determining a first version of a first segment corresponding to the first ocular zone based upon the first representation and/or the second representation; comparing a position of a first pathological feature of the one or more pathological features with a position of the first segment; and generating an updated version of the first segment based upon the comparison, wherein the ocular segmentation profile includes the updated version of the first segment.

According to some embodiments, the one or more pathological features are associated with a defined impact on one or more ocular zones; and/or one or more retinal compartment zones.

According to some embodiments, the computer-implemented method, includes analyzing the ocular segmentation profile and/or the one or more pathological features to determine one or more parameters usable for diagnosing and/or treating the person.

According to some embodiments, the computer-implemented method includes generating an ocular report indicative of the plurality of segments; the one or more pathological features; and/or the one or more parameters; and providing the ocular report to a device for display.

According to some embodiments, the one or more parameters include an ellipsoid zone (EZ) integrity.

According to some embodiments, the one or more parameters include a measure of intraretinal fluid (IRF).

According to some embodiments, the one or more parameters include a measure of subretinal fluid (SRF).

According to some embodiments, the one or more parameters include a measure of subretinal hyperreflective material (SHRM).

According to some embodiments, the one or more parameters include a measure of retinal pigment epithelium (RPE).

According to some embodiments, the one or more parameters include a measure of geographic atrophy (GA).

According to some embodiments, the one or more parameters include a measure of subretinal material (SRMat).

According to some embodiments, the one or more parameters include an ellipsoid zone (EZ) integrity; a measure of intraretinal fluid (IRF); a measure of subretinal fluid (SRF); a measure of subretinal material (SRMat); a measure of subretinal hyperreflective material (SHRM); a measure of retinal pigment epithelium (RPE); and/or a measure of geographic atrophy (GA).

According to some embodiments, the one or more pathological features include intraretinal fluid (IRF).

According to some embodiments, the one or more pathological features include subretinal fluid (SRF).

According to some embodiments, the one or more pathological features include subretinal hyperreflective material (SHRM).

According to some embodiments, the one or more pathological features include geographic atrophy (GA).

According to some embodiments, the one or more pathological features include subretinal material (SRMat).

According to some embodiments, the one or more pathological features include: intraretinal fluid (IRF); subretinal fluid (SRF); subretinal hyperreflective material (SHRM); geographic atrophy (GA); subretinal material (SRMat); drusen; cystic fluid; general fluid; and/or sub retinal pigment epithelium (RPE) fluid.

According to some embodiments, the one or more second ocular zones include the first ocular zone.

According to some embodiments, the second representation defines a region of interest between the first ocular zone and a second ocular zone of the one or more second ocular zones.

According to some embodiments, the second representation includes: a representation of the first ocular zone; and a representation of a second ocular zone of the one or more second ocular zones.

According to some embodiments, the first ocular zone corresponds to a boundary of an internal limiting membrane (ILM).

According to some embodiments, the first ocular zone corresponds to a boundary of a Bruch's membrane (BM).

According to some embodiments, the first ocular zone corresponds to a boundary of a retinal pigment epithelium (RPE).

According to some embodiments, the first ocular zone corresponds to a boundary of an outer nuclear layer (ONL).

According to some embodiments, the first ocular zone corresponds to a boundary of an ellipsoid zone (EZ).

According to some embodiments, the first ocular zone corresponds to a boundary of photoreceptor outer segments (POS).

According to some embodiments, the first ocular zone corresponds to a boundary of an external limiting membrane (ELM).

According to some embodiments, the first ocular zone corresponds to a boundary of an outer plexiform layer (OPL).

According to some embodiments, the first ocular zone corresponds to a boundary of an inner nuclear layer (INL).

According to some embodiments, the first ocular zone corresponds to a boundary of an inner plexiform layer (IPL).

According to some embodiments, the first ocular zone corresponds to a boundary of a ganglion cell layer (GCL)

According to some embodiments, the first ocular zone corresponds to a boundary of a retinal nerve fiber layer (RNFL).

According to some embodiments, the first ocular zone corresponds to a boundary of an internal limiting membrane (ILM); a Bruch's membrane (BM); a retinal pigment epithelium (RPE); an outer nuclear layer (ONL); an ellipsoid zone (EZ); photoreceptor outer segments (POS); an external limiting membrane (ELM); an outer plexiform layer (OPL); an inner nuclear layer (INL); an inner plexiform layer (IPL); a ganglion cell layer (GCL); and/or a retinal nerve fiber layer (RNFL).

According to some embodiments, an ocular zone segmentation module is provided. The ocular zone segmentation module is configured to receive one or more images of an eye of a person; use a first machine learning model to generate a first representation indicative of a first ocular zone of the eye based upon the one or more images; use a second machine learning model to generate a second representation indicative of one or more second ocular zones of the eye based upon the one or more images; and generate, based upon the first representation and the second representation, an ocular segmentation profile indicative of a plurality of segments corresponding to a plurality of ocular zones of the eye.

According to some embodiments, a system is provided. The system includes the ocular zone segmentation module; and a feature extraction module configured to identify one or more pathological features of the eye based upon the first representation, the second representation and/or the one or more images, wherein the ocular zone segmentation module generates the ocular segmentation profile based upon the one or more pathological features.

According to some embodiments, the system includes a segmentation evaluation module configured to determine a segmentation quality metric associated with the ocular segmentation profile based upon a comparison of a position of a feature of the one or more pathological features relative to a segment of the plurality of segments.

According to some embodiments, a non-transitory computer-readable medium is provided. The non-transitory computer-readable medium stores instructions that when executed perform operations including receiving one or more images of an eye of a person; using a first machine learning model to generate a first representation indicative of a first ocular zone of the eye based upon the one or more images; using a second machine learning model to generate a second representation indicative of one or more second ocular zones of the eye based upon the one or more images; and generating, based upon the first representation and the second representation, an ocular segmentation profile indicative of a first segment corresponding to the first ocular zone of the eye.

According to some embodiments, the operations include using a third machine learning model to generate a third representation indicative of the one or more second ocular zones of the eye based upon the one or more images, wherein generating the ocular segmentation profile is performed based upon the third representation.

According to some embodiments, a method including at least one aspect as described in the present disclosure and/or shown in the figures.

According to some embodiments, a method including plural aspects as described in the present disclosure and/or shown in the figures.

According to some embodiments, a system including at least one aspect as described in the present disclosure and/or shown in the figures.

According to some embodiments, a system including plural aspects as described in the present disclosure and/or shown in the figures.

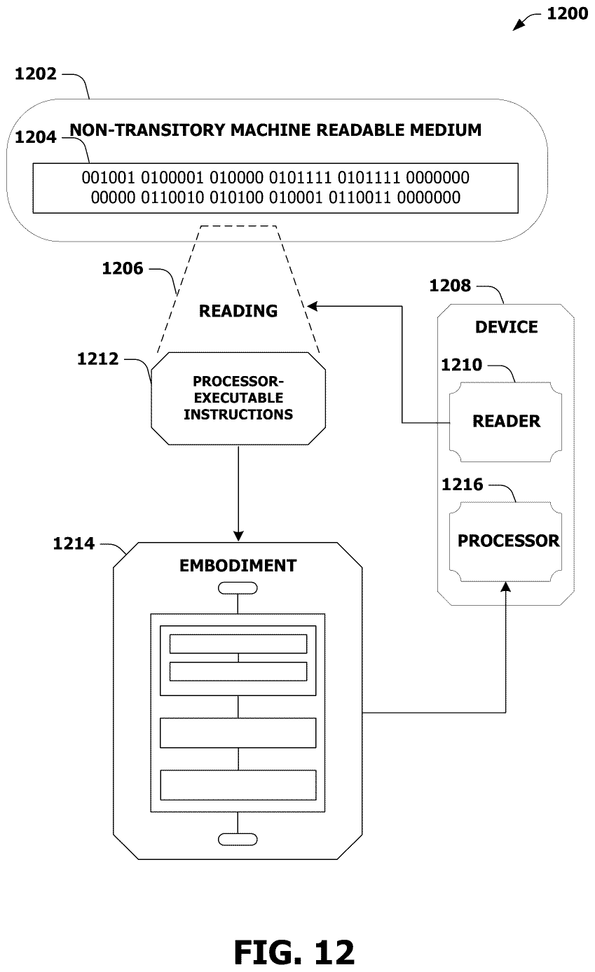

FIG. 12 is an illustration of a scenario 1200 involving an example non-transitory machine readable medium 1202. The non-transitory machine readable medium 1202 may comprise processor-executable instructions 1212 that when executed by a processor 1216 cause performance (e.g., by the processor 1216) of at least some of the provisions herein (e.g., embodiment 1214).

The non-transitory machine readable medium 1202 may comprise a memory semiconductor (e.g., a semiconductor utilizing static random access memory (SRAM), dynamic random access memory (DRAM), and/or synchronous dynamic random access memory (SDRAM) technologies), a platter of a hard disk drive, a flash memory device, or a magnetic or optical disc (such as a compact disc (CD), digital versatile disc (DVD), or floppy disk).

The example non-transitory machine readable medium 1202 stores computer-readable data 1204 that, when subjected to reading 1206 by a reader 1210 of a device 1208 (e.g., a read head of a hard disk drive, or a read operation invoked on a solid-state storage device), express the processor-executable instructions 1212.

In some embodiments, the processor-executable instructions 1212, when executed, cause performance of operations, such as at least some of the example method 1100 of FIG. 11, for example. In some embodiments, the processor-executable instructions 1212 are configured to cause implementation of a system, such as at least some of the example machine learning model training system 401 of FIGS. 4A-4C, the example system 501 of FIG. 5, for example.

As used in this application, “component,” “module,” “system”, “interface”, and/or the like are generally intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers.

Unless specified otherwise, “first,” “second,” and/or the like are not intended to imply a temporal aspect, a spatial aspect, an ordering, etc. Rather, such terms are merely used as identifiers, names, etc. for features, elements, items, etc. For example, a first object and a second object generally correspond to object A and object B or two different or two identical objects or the same object.

Moreover, “example” is used herein to mean serving as an instance, illustration, etc., and not necessarily as advantageous. As used herein, “or” is intended to mean an inclusive “or” rather than an exclusive “or”. In addition, “a” and “an” as used in this application are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, and/or variants thereof are used in either the detailed description or the claims, such terms are intended to be inclusive in a manner similar to the term “comprising”.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing at least some of the claims.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, many modifications may be made to this configuration without departing from the scope or spirit of the claimed subject matter.

Various operations of embodiments are provided herein. In an embodiment, one or more of the operations described may constitute computer readable instructions stored on one or more computer and/or machine readable media, which if executed will cause the operations to be performed. The order in which some or all of the operations are described should not be construed as to imply that these operations are necessarily order dependent. Alternative ordering will be appreciated by one skilled in the art having the benefit of this description. Further, it will be understood that not all operations are necessarily present in each embodiment provided herein. Also, it will be understood that not all operations are necessary in some embodiments.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

1. A computer-implemented method, comprising:
receiving one or more images of an eye of a person;
using a first machine learning model to generate a first representation indicative of a first ocular zone of the eye based upon the one or more images;
using a second machine learning model to generate a second representation indicative of one or more second ocular zones of the eye based upon the one or more images; and
generating, based upon the first representation and the second representation, an ocular segmentation profile indicative of a plurality of segments corresponding to a plurality of ocular zones of the eye.

2. The computer-implemented method of claim 1, comprising:
using a third machine learning model to generate a third representation indicative of the one or more second ocular zones of the eye based upon the one or more images, wherein generating the ocular segmentation profile is performed based upon the third representation.

3. The computer-implemented method of claim 2, wherein:
the one or more second ocular zones comprise the first ocular zone and a second ocular zone;
the second representation defines a region of interest between the first ocular zone and the second ocular zone; and
the third representation comprises:
a representation of the first ocular zone; and
a representation of the second ocular zone.

4. The computer-implemented method of claim 3, comprising:
identifying a plurality of images associated with a plurality of persons; and
training a machine learning model using the plurality of images and label information associated with the plurality of images to generate the second machine learning model, wherein the label information is indicative of a segment, of an image of the plurality of images, that corresponds to the region of interest between the first ocular zone and the second ocular zone.

5. The computer-implemented method of claim 3, comprising:
identifying a plurality of images associated with a plurality of persons; and
training a machine learning model using the plurality of images and label information associated with the plurality of images to generate the third machine learning model, wherein the label information is indicative of:
a first segment, of an image of the plurality of images, that corresponds to the first ocular zone; and
a second segment, of the image, that corresponds to the second ocular zone.

6. The computer-implemented method of claim 1, comprising:
identifying a plurality of images associated with a plurality of persons; and
training a machine learning model using the plurality of images and label information associated with the plurality of images to generate the first machine learning model, wherein the label information is indicative of a segment, of an image of the plurality of images, that corresponds to the first ocular zone.

7. The computer-implemented method of claim 1, comprising:
using a feature extraction machine learning model to identify one or more pathological features of the eye based upon at least one of the first representation, the second representation or the one or more images, wherein generating the ocular segmentation profile is performed based upon the one or more pathological features.

8. The computer-implemented method of claim 7, wherein generating the ocular segmentation profile comprises:
determining a first version of a first segment corresponding to the first ocular zone based upon at least one of the first representation or the second representation;
comparing a position of a first pathological feature of the one or more pathological features with a position of the first segment; and
generating an updated version of the first segment based upon the comparison, wherein the ocular segmentation profile comprises the updated version of the first segment.

9. The computer-implemented method of claim 7, wherein:
the one or more pathological features are associated with a defined impact on at least one of:
one or more ocular zones; or
one or more retinal compartment zones.

10. The computer-implemented method of claim 7, comprising:
analyzing at least one of the ocular segmentation profile or the one or more pathological features to determine one or more parameters usable for at least one of diagnosing or treating the person.

11. The computer-implemented method of claim 10, comprising:
generating an ocular report indicative of at least one of:
the plurality of segments;
the one or more pathological features; or
the one or more parameters; and
providing the ocular report to a device for display.

12-18. (canceled)

19. The computer-implemented method of claim 10, wherein:
the one or more parameters comprise at least one of:
an ellipsoid zone (EZ) integrity;
a measure of intraretinal fluid (IRF);
a measure of subretinal fluid (SRF);
a measure of subretinal material (SRMat);
a measure of subretinal hyperreflective material (SHRM);
a measure of retinal pigment epithelium (RPE); or
a measure of geographic atrophy (GA).

20-25. (canceled)

26. The computer-implemented method of claim 1, wherein:
the one or more second ocular zones comprise the first ocular zone.

27. The computer-implemented method of claim 26, wherein:
the second representation defines a region of interest between the first ocular zone and a second ocular zone of the one or more second ocular zones.

28. The computer-implemented method of claim 26, wherein:
the second representation comprises:
a representation of the first ocular zone; and
a representation of a second ocular zone of the one or more second ocular zones.

29-41. (canceled)

42. An ocular zone segmentation module configured to:
receive one or more images of an eye of a person;
use a first machine learning model to generate a first representation indicative of a first ocular zone of the eye based upon the one or more images;
use a second machine learning model to generate a second representation indicative of one or more second ocular zones of the eye based upon the one or more images; and
generate, based upon the first representation and the second representation, an ocular segmentation profile indicative of a plurality of segments corresponding to a plurality of ocular zones of the eye.

43. A system comprising:
the ocular zone segmentation module of claim 42; and
a feature extraction module configured to identify one or more pathological features of the eye based upon at least one of the first representation, the second representation or the one or more images, wherein the ocular zone segmentation module generates the ocular segmentation profile based upon the one or more pathological features.

44. The system of claim 43, comprising:
a segmentation evaluation module configured to determine a segmentation quality metric associated with the ocular segmentation profile based upon a comparison of a position of a feature of the one or more pathological features relative to a segment of the plurality of segments.

45. A non-transitory computer-readable medium storing instructions that when executed perform operations comprising:
receiving one or more images of an eye of a person;
using a first machine learning model to generate a first representation indicative of a first ocular zone of the eye based upon the one or more images;
using a second machine learning model to generate a second representation indicative of one or more second ocular zones of the eye based upon the one or more images; and
generating, based upon the first representation and the second representation, an ocular segmentation profile indicative of a first segment corresponding to the first ocular zone of the eye.

46. The non-transitory computer-readable medium of claim 45, the operations comprising:
using a third machine learning model to generate a third representation indicative of the one or more second ocular zones of the eye based upon the one or more images, wherein generating the ocular segmentation profile is performed based upon the third representation.

47-54. (canceled)
